(19) United States
(12) Patent Application Publication
(10) Pub. No.: US 2026/0364229 A1
(43) Pub. Date: Nov. 19, 2026

(54) Hierarchical Hydrogels for Tissue Regeneration

(71) Applicant: Bregen LLC, Buffalo Grove, IL (US)

(72) Inventors: Yimin WU, Buffalo Grove, IL (US); Youchao TENG, Waterloo (CA)

(73) Assignee: Bregen LLC, Buffalo Grove, IL (US)

(21) Appl. No.: 19/211,193

(22) Filed: May 18, 2025

(51) Int. Cl. A61L 27/52; A61L 27/20; A61L 27/22

(52) U.S. Cl. CPC A61L 27/52; A61L 27/20; A61L 27/222

(57) ABSTRACT

Disclosed herein provides a cytocompatible hierarchical hydrogel, which comprises a cross-linked polymer, an added cation, and an added anion. The disclosed hierarchical hydrogel composition presents at least one property having different values (anisotropic) in axis direction and transverse direction of the hydrogel, respectively. The disclosed hierarchical hydrogel is a suitable replacement for conventional hydrogels in tissue engineering, owing to their remarkable flexibility, biocompatibility, and biodegradability. The instant disclosure further relates to a method of producing the hierarchical hydrogel using ice separation-induced self-assembly approach and the spatiotemporal control of ions.

### BACKGROUND

Hydrogels have emerged as a focal point in the research for their distinctive physicochemical attributes, finding significant applications across biomedicine, tissue engineering, and soft robotics. Traditional hydrogels typically exhibit suboptimal mechanical properties, such as low fracture strength (usually less than 1 MPa), limited toughness (around 1,000 J/m²), and a low modulus (below 0.1 MPa). Constructing multiple-network hydrogels, such as double-network (DN) hydrogels, the dense networks rupture to efficiently dissipate energy, while the loose networks remain intact to withstand large strains and preserve the hydrogel's structural integrity, thereby enhancing overall mechanical performance. Despite these advancements, even with well-designed DN hydrogels, achieving toughness beyond 10 MJ·m⁻³ remains a challenge.

Alternatively, the use of covalently bonded single-network (SN) hydrogels can enhance the mechanical performance of hydrogels. While dense cross-linking in these systems enhances network stiffness, it inevitably increases brittleness. Recently, by introducing high densities of dynamic bonds (e.g., hydrogen bonding), electrostatic attraction, and host-guest interactions into SN hydrogels, or by combining structural orientation treatments (mechanically trained, fiber-filled, and freeze-cast) with solution replacement and annealing significantly enhance the mechanical properties of SN hydrogels. Current efforts of SN hydrogels primarily focus on designing heterogeneous complementary crosslinks at the molecular and nanoscale which divides labor between multiple networks or cross-linking points. Thus, it still struggles to break out of the Lake-Thomas model to reconcile the inherent trade-off between stiffness and toughness. Hence, innovative strategies are missing for the design at multiscale (i.e., micro, nano, and molecular scale) to program the mechanical properties of hydrogels due to the lack of understanding the underlying mechanisms.

Various synthetic strategies have been developed to construct hydrogels, often relying on physical crosslinking, covalent grafting, or freeze-thaw cycles. Common approaches include ice-templating to form aligned porous scaffolds, enzyme-mediated gelation to introduce dynamic interfaces, and interpenetrating polymer networks (IPNs) formed through sequential polymerization steps. Recently, solvent exchange, electrostatic layer-by-layer deposition, or gradient crosslinking have also been employed to spatially control crosslinking density in hydrogel. However, these approaches frequently fail to produce hydrogels with satisfactory mechanical properties.

### SUMMARY OF THE INVENTION

In one aspect, the present disclosure provides a hierarchical hydrogel, which comprises a cross-linked polymer, an added cation, and an added anion. Suitable cation for the hierarchical hydrogel includes H⁺, NH₄⁺, Li⁺, Na⁺, K⁺, Rb⁺, Cs⁺, Mg²⁺, Ca²⁺, Zn²⁺, Cu²⁺, Fe²⁺, Sr²⁺, Ba²⁺, Mn²⁺, Ni²⁺, Fe³⁺, Al³⁺, Cr³⁺, Ti⁴⁺, and Zr⁴⁺. Suitable anion for the hierarchical hydrogel includes OH⁻, CN⁻, SCN⁻, HCO₃⁻, HCOO⁻, CH₃COO⁻, F⁻, Cl⁻, Br⁻, I⁻, NO₃⁻, BF₄⁻, ClO₄⁻, SO₄²⁻, CO₃²⁻, SeO₄²⁻, WO₄²⁻, PO₄³⁻, SiO₄⁴⁻, Acetate (CH₃COO⁻), Propionate (C₂H₅COO⁻), Succinate (C₄H₆O₄²⁻), Fumarate (C₄H₄O₄²⁻), Oxalate (Oxalate, C₂O₄²⁻), Phosphate (H₂PO₄⁻ or HPO₄²⁻), Pyrophosphate (P₂O₇⁴⁻), Phosphonate (R—PO₃²⁻), p-Toluenesulfonic acid (TsO⁻), Methanesulfonic acid (CH₃SO₃⁻), Benzenesulfonic acid (BenzSO₃⁻), Citrate (C₆H₅O₇³⁻), Tartrate (C₄H₄O₆²⁻), Malate (C₄H₄O₅²⁻), Citrate (C₆H₅O₇³⁻), Isocitrate (C₆H₅O₇³⁻), Gluconate (C₆H₁₁O₇⁻), Oxalate (C₂O₄²⁻), Malonate (C₃H₄O₄²⁻), Succinate (C₄H₆O₄²⁻), Fumarate (C₄H₄O₄²⁻), Lactate (C₃H₅O₃⁻), Glycolate (C₂H₃O₃⁻), and Mandelate (C₆H₅CH(OH)COO⁻). The disclosed hydrogel presents at least one property having different values (anisotropic) in axis direction and transverse direction of the hydrogel, respectively. The disclosed hierarchical hydrogel is a desirable replacement for conventional hydrogels in tissue engineering, owing to their remarkable flexibility, biocompatibility, and biodegradability.

In another aspect, the present disclosure relates to a method of making the hierarchical hydrogel disclosed in the previous aspect. The disclosed method of producing the hierarchical hydrogel encompasses, for example, ice separation-induced self-assembly approach and spatiotemporal control of ions.

### BRIEF DESCRIPTION OF DRAWINGS

FIG. 1 illustrates fabrication of hierarchical structures of GE hydrogels. (a) depicts a schematic illustration of the synthesis of the AGE hydrogels combined ice separation-induced self-assembly and in-situ spatiotemporal expression programming in crystal-domain crosslinking and molecular conformation; (b) depicts a schematic diagram of the interaction structure of GE molecular chains with anions and cations; (c) depicts a schematic representation of the data assimilation framework's multi-scale objectives includes in-situ WAXS and in-situ SAXS characterization, molecular dynamics models and finite element simulation.

FIG. 2 illustrates characterization and ionic spatiotemporally programmable mechanism profiling. (a) depicts Young's modulus range of human tissues vs. GE hydrogels; (b) depicts toughness versus ultimate tensile strain of GE-SC hydrogels, other reported gelatin-based tough hydrogels and other tough materials (Gelatin/Glycerol, Gelatin/Chitosan, Gelatin/Regenerated silk fibroin, Gelatin/Ammonium sulfate, Gelatin/Potassium citrate, polydimethylsiloxane (PDMS), Polyacrylic acid (PAA), Polyvinylpyrrolidone (PVP), and Polyvinyl alcohol (PVA); (c) depicts the viscoelastic range of human tissues vs the GE hydrogels (from soft skin to hard cartilage); (d) depicts a spider web chart of GE hydrogels in terms of Young's Modulus, tensile strength, elongation, toughness, fracture energy and viscoelasticity; (e) depicts binding energy of water to SO₄²⁻, CO₃²⁻, CH₃COO⁻, C₆H₅O₇³⁻, NO₃ and Cl⁻; (f) depicts ¹H NMR spectra of water molecules in water, NaSO₄, NaCO₃, CH₃COONa, C₆H₅O₇Na₃, NaCl and NaNO₃; (g) depicts FTIR spectra of AGE-PCL, OGE, AGE-LA, AGE-PKC, AGE-PC and AGE-SC hydrogels; (h) depicts DSC spectra of AGE-PCL, OGE, AGE-LA, AGE-PKC, AGE-PC and AGE-SC hydrogels; (i) depicts WAXS plot of AGE-PCL, OGE, AGE-LA, AGE-PKC, AGE-PC and AGE-SC hydrogels.

FIG. 3 illustrates in-situ WAXS and SAXS characterization for AGE-SC hydrogels. (a) depicts 2D WAXS plot evolution of sample structure under strain; (b) depicts evolution of the angular distribution of the WAXS signal under strain; (c) depicts AGE-SC hydrogel SAXS scattering intensity plot; (d) depicts AGE-SC hydrogel SAXS and WAXS Orientation Factor curve; (e) depicts AGE-SC hydrogel in-situ SAXS stress-strain curve; (f) depicts AGE-SC hydrogel stretching molecular structure diagram.

FIG. 4 illustrates strengthening while toughening of GE-SC hydrogel. (a) depicts Cryo-SEM morphology of AGE-SC 36H hydrogel transverse direction and corresponding EDS mapping; (b) depicts Cryo-SEM morphology of AGE-SC 36H hydrogel axis direction and corresponding EDS mapping; (c) depicts the tensile stress-strain curve of the AGE-SC 36H hydrogels along the axial and transverse directions; (d) depicts Young's modulus, strength, and strain of the tested hydrogels; (e) depicts the tensile stress-strain curve of the AGE-SC 36H hydrogels along axial and transverse directions. (1 and 2) SEM images of single notched and unnotched fracture of AGE-SC 36H hydrogels on the point 1 and point 2 of the curves; (f) depicts the modulus and fracture energy of GE-SC-36H hydrogel were plotted on the stiffness-toughness plane. According to the Lake-Thomas relationship, the yellow dashed line indicates the stiffness-toughness conflict (Γ~E^{-1/2}). The AGE-SC 36H hydrogel resolves this conflict and demonstrates a coordinated enhancement of stiffness and toughness along the desired direction ((a). AGE-SC 36H hydrogel axial direction, (b). AGE-SC 36H hydrogel transverse direction, (c). AGE-SC 48H hydrogel transverse direction, (d). AGE-SC 48H hydrogel axial direction, (e). AGE-SC 24H hydrogel axial direction, (f). AGE-SC 24H hydrogel transverse direction, (g). mechanical properties of AGE-SC 36H hydrogel in the state-of-the-art ductility-modulus diagram vs previously reported high-performance hydrogels and elastomers (PVA-ANF, PCL-PNAGA, P(DMAA-co-MAAc), P(MAAm-co-MAAc), HA-PVA, P(NaSS-co MPTC), PVA sal-cryogel, Dry-annealed PVA hydrogel, COR elastomer). The yellow dash lines indicate the tradeoff between ductility and stiffness of current state-of-the-art hydrogels (Γ=240416 kN^{3/2}m⁻²×E^{-1/2}) (h) Tensile stress-strain behavior of healthy rat tendon and AGE-SC-36H samples.)

FIG. 5 depicts anisotropic hierarchical tough hydrogels (made of agar and carrageenan) by ice crystal phase separation and ion spatiotemporal programming. (a) depicts chemical structures of selected agar and carrageenan; (b, c) depict tensile stress-strain curves of agar hydrogel and carrageenan hydrogel; (d, e) depict Young's modulus and strain of agar hydrogel and carrageenan hydrogel; and (f) depicts Ashby plot of Young's modulus and ultimate tensile strain for agar and carrageenan hydrogels vs previously reported tough hydrogels (k-carrageenan (KC) hydrogel, Chitosan/Gelatin, Chitosan/alginate, Agar/Borax/Mxene, k-carrageenan/PVA, Agar-PVA/GO, k-carrageenan/Chitosan).

FIG. 6 illustrates optical and TEM images of GE solution and GE. (a) is an optical picture of GE solution; (b) depicts TEM of GE.

FIG. 7 illustrates cross-section scanning electron microscopy (SEM) images of (a, c) 15% GE-Anisotropic hydrogel and (b, d) 15% GE-Original hydrogel.

FIGS. 8(a)-(e) illustrate optical pictures and corresponding solubility of 1.5M different metal salt solutions after 36 hours; (f) illustrates dissolution of different metal salts in aqueous solution; Specifically, (f) illustrates the solubility behavior of various salts in a 1.5 M solution, highlighting the combinations of different cations (Li⁺, Na⁺, Mg²⁺, K⁺, Ca²⁺) and anions (SO₄²⁻, CO₃²⁻, CH₃COO⁻, Cl⁻, NO₃⁻, C₆H₅O₇³⁻). Generally, Na⁺ exhibits excellent solubility with all anions, forming homogeneous solutions in every case. In contrast, Li⁺, Mg²⁺, K⁺, and Ca²⁺ show varying degrees of solubility, often leading to slight precipitation with multivalent anions such as SO₄²⁻, CO₃²⁻, and C₆H₅O₇³⁻. Anions such as Cl⁻, NO₃⁻, and CH₃COO⁻ demonstrate high solubility with most cations, forming homogeneous solutions.

FIG. 9 illustrates stress-strain curves of various hydrogels. (a) AGE hydrogel and OGE hydrogel along the axial and transverse directions; (b) Stress-strain curves of AGE hydrogel after being regulated by lithium-ion metal salt solution along the axial and transverse directions; (c) Stress-strain curves of AGE hydrogel after being regulated by sodium-ion metal salt solution along the axial and transverse directions; (d) Stress-strain curves of AGE hydrogel after being regulated by magnesium-ion metal salt solution along the axial and transverse directions; (e) Stress-strain curves of AGE hydrogel after being regulated by potassium-ion metal salt solution along the axial and transverse directions; (f) Stress-strain curves of AGE hydrogel after being regulated by calcium-ion metal salt solution along the axial and transverse directions.

FIG. 10 illustrates stress-strain curves of various single notch hydrogels. (a) The tensile stress-strain curve of the single notched AGE hydrogel along the axis and transverse directions vs single notched OGE hydrogel along the axis and transverse directions; (b) Stress-strain curves of single-notched AGE hydrogel after being regulated by lithium-ion metal salt solution along the axial and transverse directions; (c) Stress-strain curves of single-notched AGE hydrogel after being regulated by sodium-ion metal salt solution along the axial and transverse directions; (d) Stress-strain curves of single-notched AGE hydrogel after being regulated by magnesium-ion metal salt solution along the axial and transverse directions; (e) Stress-strain curves of single-notched AGE hydrogel after being regulated by potassium-ion metal salt solution along the axial and transverse directions; (f) Stress-strain curves of single-notched AGE hydrogel after being regulated by calcium-ion metal salt solution along the axial and transverse directions.

FIG. 11 illustrates the stress-dependent (f=1 Hz) oscillatory profiles of GE hydrogels programmed by different metal salt solutions. (a)-(f) show the variations of storage modulus (G′) and loss modulus (G″) of different hydrogel systems under stress sweep tests. The x-axis represents stress (Pa), and the y-axis represents G′ and G″ (Pa) on a logarithmic scale. Each subfigure corresponds to different formulations, investigating the effects of various ions or additives on the mechanical properties of hydrogels. In most samples, G′ is significantly higher than G″ across the stress range of 1-100 Pa, indicating predominantly elastic behavior.

FIG. 12 illustrates the frequency-dependent (at stress of 1 Pa) oscillatory profiles of GE hydrogels programmed by different metal salt solutions. a-f, Storage modulus (G′) and loss modulus (G″) as a function of frequency (0.1-16 Hz) for hydrogels with different formulations. In all cases, G′ is higher than G″, indicating elastic-dominated behavior. The magnitude and frequency dependence of the moduli vary with formulation, reflecting differences in network strength and viscoelasticity.

FIG. 13 illustrates the frequency dependence of the loss tangent (tan δ) of GE hydrogels programmed by different metal salt solutions. (a)-(f), Evolution of the loss factor (tan δ, defined as G″/G′) as a function of frequency (0.1-16 Hz) for hydrogels incorporating various ions or additives. In all cases, tan δ remains low across the frequency range, indicating elastic-dominated behavior. Variations in tan δ values among different formulations reflect differences in network viscoelasticity and energy dissipation characteristics.

FIG. 14 illustrates the viscosity of GE hydrogels is programmed by different metal salt solutions at an increased shear rate, followed by a reverse shear rate decrease in continuous flow experiments. (a)-(f), Viscosity as a function of shear rate (0.1-9 s⁻¹) for hydrogels incorporating various ions or additives. All samples exhibit shear-thinning behavior, as evidenced by the continuous decrease in viscosity with increasing shear rate. The magnitude of viscosity and the extent of shear-thinning vary among different formulations, reflecting differences in network structure and flow resistance.

FIGS. 15 (a)-(e) illustrate FTIR spectra of AGE hydrogels programmed by different metal salt solutions; (f) illustrates FTIR spectra of the OGE and AGE hydrogel. Characteristic absorption bands associated with gelatin and introduced functional groups are observed across all samples. Comparisons reveal formulation-dependent variations in the intensities and positions of amide I, amide II, and other relevant peaks, indicating chemical modifications of the hydrogel networks. f, Comparison between original gelatin hydrogel (OGE) and modified gelatin hydrogel (AGE), demonstrating the introduction of new functional groups after modification.

FIGS. 16 (a)-(e) illustrate DSC spectra of GE hydrogels programmed by different metal salt solutions; (f) illustrates DSC spectra of the original and directed freezing GE hydrogel. Endothermic transitions corresponding to the melting temperature (Tₘ) are observed in all samples. The position and magnitude of Tₘ vary with formulation, indicating differences in thermal stability and network interactions. f, Comparison between original (OGE) and modified (AGE) hydrogels, demonstrating an increase in Tₘ after modification, suggesting enhanced network cohesion.

FIGS. 17 (a)-(e) illustrates WAXS spectra of GE hydrogels programmed by different metal salt solutions; (f) illustrates WAXS spectra of the original and directed freezing GE hydrogel. All samples display a broad scattering peak, indicative of partially ordered nanostructures. Shifts in peak position and changes in scattering intensity among different formulations reflect variations in network architecture and characteristic domain spacing. f, Comparison between original gelatin hydrogel (OGE) and modified gelatin hydrogel (AGE), showing enhanced nanoscale ordering after modification.

FIG. 18 illustrates hydrogen bonds form between GE chains induced by anions and cations due to the ionic spatiotemporal programming. Left, Anions (SO₄²⁻, CO₃²⁻, and C₂H₃O₂⁻) released from the hydrogel diffuse toward the substrate surface, modulating interfacial interactions and potentially disrupting adhesion. Right, Multivalent cations (Li⁺, Ca²⁺, and Mg²⁺) form strong ionic crosslinks between the hydrogel and the substrate, enhancing interfacial adhesion and structural integrity.

FIG. 19 illustrates mechanical properties of the 15% GE hydrogel programmed by lithium-ion metal salt solution, measured by tensile experiments and numerical simulations. (a) The strain-stress curves of the hydrogel along the longitudinal direction from experiments and simulations; (b) Young's modulus of the hydrogel along the transverse direction from experiments (

$${E}_{22}^{t}$$

).

FIG. 20 illustrates mechanical properties of the hydrogel material 15% GE hydrogel programmed by sodium-ion metal salt solution, measured by tensile experiments and numerical simulations. (a) illustrates the strain-stress curves of the hydrogel along the longitudinal direction from experiments and simulations; (b) Young's modulus of the hydrogel along the transverse direction from experiments (

$${E}_{22}^{t}$$

).

FIG. 21 illustrates mechanical properties of the hydrogel material 15% GE hydrogel programmed by magnesium-ion metal salt solution, measured by tensile experiments and numerical simulations. (a) The strain-stress curves of the hydrogel along the longitudinal direction from experiments and simulations; (b) Young's modulus of the hydrogel along the transverse direction from experiments (

$${E}_{22}^{t}$$

).

FIG. 22 illustrates mechanical properties of the hydrogel material 15% GE hydrogel programmed by potassium-ion metal salt solution, measured by tensile experiments and numerical simulations. (a) The strain-stress curves of the hydrogel along the longitudinal direction from experiments and simulations; (b) Young's modulus of the hydrogel along the transverse direction from experiments (

$${E}_{22}^{t}$$

).

FIG. 23 illustrates mechanical properties of the hydrogel material 15% GE hydrogel programmed by calcium-ion metal salt solution, measured by tensile experiments and numerical simulations. (a) The strain-stress curves of the hydrogel along the longitudinal direction from experiments and simulations; (b) Young's modulus of the hydrogel along the transverse direction from experiments (

$${E}_{22}^{t}$$

).

FIG. 24 illustrates mechanical properties of the hydrogel material OGE, measured by tensile experiments and numerical simulations. (a) The strain-stress curves of the hydrogel along the longitudinal direction from experiments and simulations; (b) Young's modulus of the hydrogel along the transverse direction from experiments (

$${E}_{22}^{t}$$

).

FIG. 25 illustrates the tensile stress-strain curve of the unnotched and single-notched AGE-SC hydrogels along the axis and transverse directions. Tensile stress-strain behavior of AGE-SC hydrogels after 24, 36, and 48 hours of treatment, tested along the transverse and axial directions, with and without a single notch. Hydrogels exhibit distinct mechanical responses depending on the orientation, treatment time, and presence of a notch, indicating the evolution of anisotropic network structures and fracture resistance.

FIG. 26 illustrates SEM morphology of AGE hydrogel after being regulated by lithium-ion metal salt solution. (a)-(d), Representative SEM images of AGE-LS (a), AGE-LKC (b), AGE-LA (c), and AGE-LC (d) hydrogels, highlighting their interconnected porous architectures. The width of the pores and the thickness of the pore walls are indicated by yellow arrows. Variations in pore size and wall thickness among different formulations reflect differences in network density and microstructural organization.

FIG. 27 illustrates SEM morphology of AGE hydrogel after being regulated by sodium-ion metal salt solution. (a)-(e), Representative SEM images of AGE-SS (a), AGE-SKC (b), AGE-SA (c), AGE-SCL (d), and AGE-SC (e) hydrogels, highlighting their interconnected porous architectures. Pore widths and wall thicknesses are indicated by yellow arrows. Differences in pore size and wall structure among the samples reflect variations in network morphology and microstructural densification.

FIG. 28 illustrates SEM morphology of AGE hydrogel after being regulated by magnesium-ion metal salt solution. (a)-(d), Representative SEM images of AGE-MS (a), AGE-MKC (b), AGE-MA (c), and AGE-MC (d) hydrogels, highlighting their interconnected porous architectures. The width of the pores and the thickness of the pore walls are indicated by yellow arrows. Notable differences in pore size and wall thickness among the samples reflect formulation-dependent variations in network morphology and hierarchical structuring.

FIG. 29 illustrates SEM morphology of AGE hydrogel after being regulated by potassium-ion metal salt solution. (a)-(e), Representative SEM images of AGE-PS (a), AGE-PKC (b), AGE-PA (c), AGE-PCL (d), and AGE-PC (e) hydrogels, highlighting their interconnected porous networks. Pore width and wall thickness are indicated by yellow arrows. Differences in pore morphology and wall dimensions among the samples reflect variations in microstructural organization and network densification.

FIG. 30 illustrates SEM morphology of AGE hydrogel after being regulated by calcium-ion metal salt solution (including pore size and pore wall thickness). (a)-(d), Representative SEM images of AGE-CS (a), AGE-CKC (b), AGE-CA (c), and AGE-CC (d) hydrogels, highlighting their well-defined porous architectures. Pore width and wall thickness are indicated by yellow arrows. Differences in pore dimensions and structural organization reflect variations in network formation and mechanical reinforcement among different formulations.

FIG. 31 illustrate the diagonal width of the holes in microfiber structure and the thickness of the microfiber structure, measured by SEM. (a) Comparison of pore width and wall thickness between AGE and OGE hydrogels. (b)-(f), Quantitative comparison among different AGE hydrogel formulations, illustrating formulation-dependent modulation of pore dimensions. Orange bars represent average pore widths; green bars represent average wall thicknesses. Error bars denote standard deviations.

### DETAILED DESCRIPTION OF INVENTION

Conventional hydrogels, such as hydrogels made from gelatin (GE) and polysaccharides, have been extensively employed to mimic the extracellular matrix (ECM), supporting cell growth, migration, and differentiation, or to construct human-like soft tissue structures. However, conventional hydrogels often achieve only a limited range of mechanical property modulation and rely on complex preparation processes, which restrict their practical applications. For instance, in tendon repair, the mechanical properties of conventional GE hydrogels reported in existing studies are typically below 7 MPa, substantially lower than the strength of natural tendons (~13 MPa), and far from meeting the performance standards of tough hydrogels. These limitations are primarily attributed to the lack of hierarchical structure and effective cross-linking strategies.

To address these limitations, it is imperative to further optimize the mechanical properties of conventional hydrogels to satisfy the demands of tissue engineering and related fields. Conventional hydrogels could incorporate dynamic bonds or hierarchical structures to enhance their mechanical performance. A multiscale hierarchical design strategy that regulates interactions between hydrogel polymer chains at the molecular scale while optimizing the internal structural orientation of hydrogels at the macroscopic scale could enable a wide range of tunable mechanical properties without compromising their intrinsic advantages, such as flexibility and biocompatibility.

The current disclosure provides a novel hierarchical hydrogel characterized by multiscale structuring—molecular, nano- to micro-scale domains—that contributes synergistically to enhanced mechanical, functional, or biological performance. In particular, the hierarchical hydrogel disclosed herein shows a variety of anisotropic properties. Hierarchical hydrogels in the current disclosure hold significant potential as a replacement for conventional hydrogels in tissue engineering, owing to their remarkable flexibility, biocompatibility, and biodegradability.

Anisotropic properties of the hierarchical hydrogel can be characterized by two principal directions: the axial (longitudinal) direction and the transverse direction. The axial direction represents the primary alignment or growth direction of the hydrogel structure, typically following along the major elongation, stretching, or processing axis. In fiber- or layered-based hydrogels, this direction runs parallel to fiber or polymer chain alignment. In molded hydrogels, it is often determined by the casting process or flow-induced molecular orientation. For example, in extruded or directionally aligned hydrogels, the axial direction follows the longitudinal pathway of extrusion. In contrast, the transverse direction is oriented perpendicular to the axial alignment. In fiber- or layered-based hydrogels, it is perpendicular to polymer or fiber alignment, whereas in molded hydrogels, it typically corresponds to the material's width or thickness. This distinction between axial and transverse directions is critical for understanding and optimizing the mechanical and structural behavior of hierarchical hydrogels in various applications.

Suitable polymers for constructing hierarchical hydrogel include three distinct categories of materials: gelatin, plant polysaccharides, and animal polysaccharides.

(a) Gelatin-based materials (GE). GE is generally derived from partial hydrolysis of collagen. Gelatin offers excellent biocompatibility, tunable gelation properties, and abundant functional groups suitable for crosslinking or ionic modulation. It serves as a versatile base matrix in many hierarchical hydrogel systems. (b) Plant polysaccharides. Naturally occurring biopolymers include cellulose, nanocellulose, alginate, starch derivatives, and carrageenan. (c) Animal polysaccharides. Animal polysaccharides are generally derived from marine or terrestrial animal sources, including chitosan, hyaluronic acid (hydrophilic, viscoelastic, cell-interactive), heparin. These materials serve as structural and functional building blocks, often combined or sequentially crosslinked to form multiscale porous networks, interpenetrating domains, or ionic microenvironments. The resultant hierarchical architecture can be tailored for specific mechanical, transport, or biological functions.

The current disclosure further discloses a method of making the hierarchical hydrogels. The method generally involves preparation of hydrogel polymer (e.g. gelatins, polysaccharides, or combination of various types of polymers) solutions, preparation of salt solutions, and production of hierarchical hydrogels. Specifically, a hydrogel polymer solution was prepared by dissolving the polymer powder in water with vigorous stirring and heating. After degassing, a clear polymer solution was obtained. Different metal salt solutions were prepared by dissolving corresponding salt powders in water. Proper treatment was applied to obtain a clear solution. Hierarchical hydrogels were subsequently produced under cryogenic conditions. For example, a liquid nitrogen bath was used as the immersion bath for the ice template. 3D-printed homemade molds were used as containers. For the typical fabrication of layered hierarchical hydrogels, an aqueous polymer precursor was poured into a homemade template with a metal sheet (such as copper sheet) attached to the bottom of container to achieve good thermal conductivity. Subsequently, the container was placed into a liquid nitrogen bath at a controlled immersion rate. The directionally frozen polymer solution was then immersed in each of the above-mentioned salts solutions for gelation at varying times as materials required for subsequent studies.

In one aspect, the instant disclosure relates to a biocompatible hierarchical hydrogel with widely programmable mechanical properties (such as Young's modulus, viscoelasticity, tensile strain, and toughness) that mimic and match the complex functions of natural biological tissues.

In one embodiment, the hierarchical hydrogel comprises a cross-linked polymer, an added cation, and an added anion. Suitable cross-linked polymers for constructing the hierarchical hydrogel include at least gelatin, plant polysaccharides, animal polysaccharides or a combination of two or more of these polymers.

In the same embodiment, gelatin polymer is generally derived from partial hydrolysis of collagen. Plant polysaccharides include naturally occurring biopolymers such as cellulose, nanocellulose, alginate, starch derivatives, and carrageenan. Animal polysaccharides are generally derived from marine or terrestrial animal sources, including at least: chitosan, hyaluronic acid, and heparin.

In the same embodiment of the hierarchical hydrogel, suitable cation is selected from the group consisting of H⁺, NH₄⁺, Li⁺, Na⁺, K⁺, Rb⁺, Cs⁺, Mg²⁺, Ca²⁺, Zn²⁺, Cu²⁺, Fe²⁺, Sr²⁺, Ba²⁺, Mn²⁺, Ni²⁺, Fe³⁺, Al³⁺, Cr³⁺, Ti⁴⁺, and Zr⁴⁺. In the embodiment, suitable anion is selected from the group consisting of OH⁻, CN⁻, SCN⁻, HCO₃⁻, HCOO⁻, CH₃COO⁻, F⁻, Cl⁻, Br⁻, I⁻, NO₃⁻, BF₄⁻, ClO₄⁻, SO₄²⁻, CO₃²⁻, SeO₄²⁻, WO₄²⁻, PO₄³⁻, SiO₄⁴⁻, Acetate (CH₃COO⁻), Propionate (C₂H₅COO⁻), Succinate (C₄H₆O₄²⁻), Fumarate (C₄H₄O₄²⁻), Oxalate (Oxalate, C₂O₄²⁻), Phosphate (H₂PO₄⁻ or HPO₄²⁻), Pyrophosphate (P₂O₇⁴⁻), Phosphonate (R—PO₃²⁻), p-Toluenesulfonic acid (TsO⁻), Methanesulfonic acid (CH₃SO₃⁻), Benzenesulfonic acid (BenzSO₃⁻), Citrate (C₆H₅O₇³⁻), Tartrate (C₄H₄O₆²⁻), Malate (C₄H₄O₅²⁻), Citrate (C₆H₅O₇³⁻), Isocitrate (C₆H₅O₇³⁻), Gluconate (C₆H₁₁O₇⁻), Oxalate (C₂O₄²⁻), Malonate (C₃H₄O₄²⁻), Succinate (C₄H₆O₄²⁻), Fumarate (C₄H₄O₄²⁻), Lactate (C₃H₅O₃⁻), Glycolate (C₂H₃O₃⁻), and Mandelate (C₆H₅CH(OH)COO⁻). In a further embodiment, the added cation is Li⁺, Na⁺, Mg²⁺, K⁺, Ca²⁺, or a combination thereof; and the added anion is SO₄²⁻, CO₃²⁻, CH₃COO⁻, Cl⁻, NO₃⁻, C₆H₅O₇³⁻, or a combination thereof.

In the same embodiment, the disclosed hierarchical hydrogel exhibits at least one anisotropic property, where the property presents different values (anisotropic) in axis direction and transverse direction of the hydrogel. Such properties of the hierarchical hydrogel may include, but not limited to tensile strength, Young's Modulus (Elastic Modulus, Pa), fracture toughness, fracture energy, and viscoelastic properties (Storage Modulus G′ and Loss Modulus G″), pore alignment, fibril orientation/polymer chain orientation, crystallinity, and tensile strain.

Tensile Strength generally refers to the maximum stress a material can endure while being stretched or pulled before it breaks. In engineering, it is quantified as the force per unit area (usually in megapascals, MPa) at which a material sample fails during a tensile test. In a further embodiment of this aspect, tensile strength of the hierarchical hydrogel varies between about 1 kPa and about 10 MPa (including, for example, about 1, 2, 3, 4, 5, 6, 7, 8, 9, 10, 50, 100, 500, 900 kPa and about 1, 2, 3, 4, 5, 6, 7, 8, 9, 10 MPa) in transverse direction, and between about 1 KPa and about 100 MPa (including, for example, about 1, 2, 3, 4, 5, 6, 7, 8, 9, 10, 50, 100, 500, 900 kPa and about 1, 2, 3, 4, 5, 6, 7, 8, 9, 10, 20, 50, 100 MPa) in axial direction. In a preferred embodiment, tensile strength of the hierarchical hydrogel varies between 6±1 kPa and 4.84±1 MPa in transverse direction, between 7±1 kPa and 14.25±1 MPa in axial direction.

Young's Modulus (Elastic Modulus, Pa) quantifies the stiffness of an elastic material. It is defined as the ratio of tensile stress (a) to tensile strain (E) within the linear elastic region of the stress-strain curve during a tensile test. Expressed in pascals (Pa), it indicates the material's capacity to deform elastically (i.e., without permanent deformation). In a further embodiment of this aspect, Young's Modulus of the hierarchical hydrogel varies between about 1 kPa and about 5 MPa (including, for example, about 1, 2, 3, 4, 5, 6, 7, 8, 9, 10, 50, 100, 500, 900 kPa and about 1, 2, 3, 4, 5 MPa) in transverse direction, and between about 10 kPa and about 200 MPa (including, for example, about 10, 20, 50, 100, 500, 900 kPa and about 1, 2, 3, 4, 5, 6, 7, 8, 9, 10, 50, 100, 200 MPa) in axial direction. In a preferred embodiment, Young's Modulus of the hierarchical hydrogel varies between 9.7±0.2 kPa and 2.407±1 MPa in transverse direction, and between 20 kPa±10 kPa and 3.595±0.120 MPa in axial direction.

Fracture Toughness describes a material's ability to resist further fracture when a crack is present. It represents the critical value of the stress intensity factor at the crack tip when the crack propagates suddenly. This parameter is essential for predicting the service life of a material under stress and in the presence of flaws. In a further embodiment of this aspect, fracture toughness of the hierarchical hydrogel varies between about 0.0005 MJ·m⁻³ and about 5 MJ·m⁻³ (including, for example, about 0.0005, 0.001, 0.005, 0.01, 0.05, 0.1, 0.5, 1, 2, 3, 4, and 5 MJ·m⁻³) in transverse direction, and between about 0.001 MJ·m⁻³ and about 200 MJ·m⁻³ (including, for example, about 0.001, 0.005, 0.01, 0.05, 0.1, 0.5, 1, 2, 3, 4, 5, 10, 20, 50, 100, and 200 MJ·m⁻³) in axial direction. In a preferred embodiment, fracture toughness of the hierarchical hydrogel varies between 0.00127±0.0005 MJ·m⁻³ and 3.43±0.5 MJ·m⁻³ in transverse direction, and between 0.0032±0.0005 MJ·m⁻³ and 28.89±0.2 MJ·m⁻³ in axial direction.

Fracture energy is the amount of energy absorbed per unit area by a material as it fractures. The energy absorption during fracture significantly influences the material's behavior underload. In a further embodiment of this aspect, fracture energy of the hierarchical hydrogel varies between about 0.00001 MJ/m² and about 1 MJ/m² (including, for example, about 0.00001, 0.00005, 0.0001, 0.0005, 0.001, 0.005, 0.01, 0.02, 0.05, 0.1, 0.2, 0.5, 0.8, and 1 MJ/m²) in transverse direction, and between about 0.0001 MJ/m² and about 20 MJ/m² (including, for example, about 0.0001, 0.0005, 0.001, 0.005, 0.01, 0.05, 0.1, 0.2, 0.3, 0.4, 0.5, 1, 2, 5, 10, and 20 MJ/m²) in axial direction. In a preferred embodiment, fracture energy of the hierarchical hydrogel varies between about 65.8 J·m⁻² and about 66986.1 J·m⁻² in transverse direction, and between about 132.1 J·m⁻² and about 413304.7 J·m⁻² in axial direction.

Viscoelastic properties are time-dependent mechanical response of the hydrogel may vary depending on the direction of applied stress. In a further embodiment of this aspect, viscoelasticity of the hierarchical hydrogel varies between about 0.001 and about 0.5 (including, for example, about 0.001, 0.002, 0.005, 0.01, 0.02, 0.05, 0.1, 0.2, 0.3, 0.4, and 0.5) in transverse direction, and between about 0.002 and about 0.5 (including, for example, about 0.002, 0.005, 0.01, 0.02, 0.05, 0.1, 0.2, 0.3, 0.4, and 0.5) in axial direction. In a preferred embodiment, viscoelasticity of the hierarchical hydrogel varies between 0.0057±0.0012 and 0.2465±0.025 in transverse direction, and between 0.0657±0.0012 and 0.3867±0.016 in axial direction.

In a further embodiment of this aspect, the hierarchical hydrogel exhibits average pore size ranging from 0.53±0.15 μm to 5.53±0.5 μm (including, for example, about 0.53, 1.00, 1.50, 2.00, 3.00, 4.00, 5.00, and 5.53 μm) in transverse direction and from 1.42±0.23 μm to 10.91±1.96 μm (including, for example, about 1.42, 1.50, 2.00, 3.00, 4.00, 5.00, 6.00, 7.00, 8.00, 9.00, 10.00, and 10.91 μm) in axial direction.

Porosity measures the void spaces (or pores) within a material, typically expressed as a percentage of the total volume occupied by these void spaces. It indicates the amount of empty (or void) space within a solid material, affecting properties such as strength, permeability, and density. In a further embodiment of this aspect, crystallinity of the hierarchical hydrogel varies between about 5% and about 9.32% (including, for example, about 5.00%, 5.50%, 6.00%, 7.00%, 8.00%, 9.00%, and 9.32%) in transverse direction, and between about 30.33% and about 70% (including, for example, about 30.33%, 35.00%, 40.00%, 50.00%, 60.00%, and 70.00%) in axial direction.

Pore alignment and porosity are defined as the directional alignment of pores within the hydrogel's porous structure, which affects diffusion rates and/or mechanical properties of the hydrogel. Pores can be randomly distributed or aligned in a specific direction. Pore alignment can significantly influence a material's mechanical properties, such as anisotropy in mechanical strength or directional permeability. In certain embodiments of this aspect, the pores are all oriented, but the porosity varies due to the modulation of different salt ions. In these embodiments, scanning electron microscopy (SEM) images to measure the pore size. In a further embodiment of this aspect, porosity of the hierarchical hydrogel ranges between about 10% and about 50% (including, for example, about 10%, 15%, 20%, 25%, 30%, 35%, 40%, 45%, and 50%) in transverse direction, and between about 20% and about 90% (including, for example, about 20%, 25%, 30%, 35%, 40%, 45%, 50%, 55%, 60%, 65%, 70%, 75%, 80%, 85%, and 90%) in axial direction.

Fibril or polymer chain orientation refers to the alignment of polymer chains or gelatin fibrils within the hydrogel, resulting in anisotropic mechanical responses. Fibrils are small, thread-like structures within fibrous materials. The orientation of these fibrils significantly impacts on the material's mechanical properties, including tensile strength and elasticity. In biological tissues such as collagen and muscle, fibril orientation plays a critical role in ensuring optimal functional performance. Fibril orientation/polymer chain orientation is characterized by inter-chain arrangement Herman orientation factor. In a further embodiment of this aspect, Herman orientation factor of the hierarchical hydrogel ranges between about 0.50 and about 0.55 (including, for example, about 0.50, 0.51, 0.52, 0.53, 0.54, and 0.55) in transverse direction, and between about 0.65 and about 0.70 (including, for example, about 0.65, 0.66, 0.67, 0.68, 0.69, and 0.70) in axial direction.

Tensile strain is the relative deformation of a material when subjected to uniaxial tensile loading. It is defined as the change in length divided by the original length. In a further embodiment of this aspect, tensile strain of the hierarchical hydrogel ranges between about 10% and about 200% (including, for example, about 10%, 15%, 20%, 30%, 40%, 50%, 60%, 70%, 80, 90%, 100%, 150%, and 200%) in transverse direction, and between about 50% and about 500% (including, for example, about 50%, 60%, 70%, 80, 90%, 100%, 150%, 200%, 250%, 300%, 350%, 400%, 450%, and 500%) in axial direction.

In further embodiments of this aspect, the hierarchical hydrogel exhibits water content ranging from about 70% to about 90%, including, for example, about 70%, 75%, 80%, 85%, and 90%.

In one aspect, the instant disclosure provides a method that leverages the synergistic effect of ice separation-induced self-assembly and ion spatiotemporal expression to orchestrate the programming of molecular chain conformation and pore structure to prepare cytocompatible hierarchical hydrogel with accurate and broad modulation of mechanical properties. The synergistic effect of ice separation-induced self-assembly and ion spatiotemporal expression was utilized to orchestrate the programming of molecular chain conformation and pore structure to prepare cytocompatible hierarchical hydrogel with accurate and broad modulation of mechanical properties.

Hereinafter, the present invention will be described in more detail by way of Examples. The invention will be more readily understood by reference to the following examples. However, the technical scope of the present invention is not intended to be limited only to the following Examples.

### EXAMPLES

### Example 1. Preparation of Hierarchical GE Hydrogel (or GE Hydrogel)

Preparation of GE Solution. A 15 wt. % GE solution was prepared by dissolving GE powder in deionized water with vigorous stirring and heating (55 °C). After ultrasonic degassing for 1 hour, a clear solution was obtained.

Preparation for Salt Solution. At 1.5M 30 different metal salt solutions were prepared by dissolving various metal salt powders in deionized water. After sonication for 10 minutes, a clear solution was obtained.

Preparation of GE Hydrogel. A liquid nitrogen bath was used as the immersion bath for the ice template. 3D-printed homemade molds were used as containers. For the typical fabrication of layered GE hydrogels, 15 wt. % aqueous GE precursor was poured into a homemade template with a copper sheet attached to the bottom to achieve good thermal conductivity. Then, the container was placed into a liquid nitrogen bath at a 2 mm mini-immersion rate. The directionally frozen GE solution was then immersed in 1.5 M of different metal salts for gelation at varying times as materials required for subsequent studies.

### Example 2. Evaluation of Properties of GE Hydrogels Obtained in Example 1

### 1. Tensile Test of GE Hydrogel Prepared in Example 1.

The rectangular hydrogels with a width of 20 mm, a thickness of 2 mm, and a height of 40 mm for routine tensile testing. The initial clamping distance (the sample distance between the clamps) for each pair of samples was from 1 cm to 2 mm. Tests were conducted from the oriented direction along the anisotropic structure and from the non-oriented direction. Force-displacement data were obtained using a universal mechanical tester with 50 N and 200 N loading cells. Stress, denoted as σ (MPa), was calculated by dividing the measured force F (N) by the initial gauge cross-sectional area A (m²), as described in Equation 1.

$$σ = \frac{F}{A} \qquad ( 1 )$$

Strain (ε) was determined by dividing the measured displacement Lₘ (mm) by the initial clamping distance L₀ (mm), as described in Equation 2.

$$ε \frac{{L}_{m}}{{L}_{0}} \qquad ( 2 )$$

Five hydrogel samples were tested under each condition.

### 2. Fracture Mechanic's Test

For characterization, the fracture behavior of GE hydrogels of Example 1—both notched and unnotched rectangular hydrogels with a width of 20 mm, a thickness of 2 mm, and a height of 40 mm was tested as a pair to measure the fracture toughness. The initial clamping distance (the sample distance between the clamps) for each pair of samples from 1 cm to 2 mm. All samples have microstructural alignment parallel to the height direction. For fracture mechanics, two identical specimens (one notched, one unnotched) were loaded as a pair at sample settings to obtain a fracture energy value. Briefly, the notched samples with a 8-mm-long cut in the middle of the edge were first stretched at a speed of 60 mm/min to acquire the stress curves, in which the critical strain F, referring to the strain at crack propagation was obtained. The pairing unnotched samples were also tested to reach critical strain. The toughness of hydrogel was calculated by integrating the area beneath the stress-strain curves of unnotched specimens from the start point to the critical strain point (ε_{c}), as formula

$$Toughness = {\int }_{0}^{{ε}_{c}} σ d ε \qquad ( 3 )$$

The fracture energy value was obtained by multiplying the area under the stress-strain curve of the unnotched specimens with the initial clamp distance (H) as

$$τ = H {\int }_{0}^{{ε}_{c}} σ d ε \qquad ( 4 )$$

### 3. SEM Characterization

To characterize the microstructure and nanostructure of hierarchically aligned hydrogels, they were freeze-dried using a Labconco FreeZone freeze dryer. The lyophilized hydrogel was cut along the alignment direction to expose its interior, sputtered with gold, and then imaged using a Hitachi SU5000.

### 4. Cryo-SEM Characterization

To characterize the microstructure and nanostructure of hierarchically aligned sodium citrate GE hydrogel. They were cut along the alignment direction to expose its interior, sputtered with gold, and then imaged using a Zeiss Crossbeam 350.

### 5. Moisture Content Measurement

Water content of GE hydrogels was measured by comparing their weights before and after freeze-drying. All GE hydrogel samples (3 mm (L)×3 mm (W)×1 mm (t)) were immediately frozen using liquid nitrogen, followed by vacuum freeze drying to ensure complete removal of water molecules. The water content (Øwater) of the GE hydrogels was determined as follows:

$$∅ water = \frac{{m}_{wet} - {m}_{dry}}{{m}_{wet}} \times 100 \% \qquad ( 5 )$$

### 6. Crystallinity Content Measurement

The crystallinities of all dry-state GE hydrogels were quantified by a Differential Scanning Calorimetry (DSC, TA), as previously reported.^{[11]} All the GE hydrogels were first chemically crosslinked with excessive glutaraldehyde, to fix the amorphous polymer chains before air-drying, thus no further crystallization will occur during the drying process. All the GE hydrogel samples were immediately frozen using liquid nitrogen, followed by vacuum freeze drying to ensure complete removal of water molecules before the DSC test. In the DSC test, the dried samples were heated up from 25 °C to 250 °C at a rate of 10 °C min⁻¹, under a nitrogen flow of 35 mL min⁻¹. The mass of crystalline domains (m_{crystalline}) and the total mass of the dry samples (mₜₒₜₐₗ) were measured by DSC measurements. The enthalpy required for melting the crystalline domains per unit mass of the dry sample H_{crystalline} can be estimated by integrating the endothermic transition ranging of each sample. Therefore, the mass of the crystalline domains (m_{crystalline}) can be calculated as

$${m}_{crystalline} = {m}_{total} \times \frac{{H}_{crystalline}}{{H}_{crystalline}^{0}} \qquad ( 6 )$$

where H⁰_{crystalline}=62.5 J g⁻¹ is the enthalpy of melting of 100% crystalline protein.⁵⁰ The crystallinity of dry samples X_{dry} can be calculated as

$${X}_{dry} = \frac{{m}_{crystalline}}{{m}_{total}} \times 100 \% \qquad ( 7 )$$

For each type of hydrogel materials, three independent samples were measured.

### 7. FTIR Measurement

Attenuated total reflectance FTIR spectroscopy (Agilent CARY 660) was conducted to characterize the specific chemical groups in the hydrogels. All GE Hydrogels were cut as 1-mm-thick films and then tested in the range of wave number from 4000 to 400 cm⁻¹.

### 8. NMR Test

NMR analysis was carried out on an AVANCE III 400 MHz equipment (Switzerland) to further illustrate the H bonds changes in various metal salt solution. A liquid sample of 400 μL was measured via the coaxial double tube where the inner tube was filled with deuterated reagent and the outer tube was filled with the sample. The deuterated reagent was used as the field frequency lock for NMR tests. Deuterated H₂O (D₂O), a common reagent, was used for room-temperature NMR test. For the NMR tubes in gel solution, all samples were heated to 40 °C in the machine and held for 10 minutes before starting the measurements.

### 9. Small- and Wide-Angle X-Ray Scattering (SAXS/WAXS)

The small- and wide-angle X-ray scattering (SAXS/WAXS) experiments were conducted at the Complex Material Scattering (CMS, 11-BM) beamline at the National Synchrotron Light Source II (NSLS-II) at Brookhaven National Laboratory (BNL). The incident X-ray beam used in this experiment had an energy of 13.5 keV, with a corresponding wavelength of λ=0.9184 Λ, with an energy resolution of ΔE/E=0.7%. A focused beam size of 0.2 mm×0.2 mm was employed, with a photon flux of approximately 10¹² photons/s, ensuring sufficient signal for both static (ex-situ) and dynamic (in-situ) measurements. The scattering signal was captured using two dedicated detectors: a customized Pilatus 800K detector for WAXS data acquisition, positioned at a sample-to-detector distance of 0.260 m, and a Pilatus 2M detector for SAXS, located at 5 m. This setup allowed capturing structural information at different length scales in concurrent SAXS/WAXS measurements.

For ex-situ (static) SAXS/WAXS measurements, samples were mounted on a standardized custom multiple-sample holder, with an exposure time set to 30 s per measurement to capture high-resolution structural data. For in-situ (tensile) measurements, samples were mounted on a Linkam MFS (Modular Force Stage) equipped with a Linkam T96-S Tensile Controller. The initial gauge length between clamps was set to Lₓ₀≈17.5 mm, with tensile stretching speed applied at a constant rate of ΔLₓ=100 μm/s. To capture the structural evolution during stretching, SAXS/WAXS data were collected at 20 s intervals, each with a 10 s exposure time, thus providing a time-resolved view of the structural response under stress. Stress and strain in the in-situ tensile experiments were calculated under the assumption of volume conservation, expressed as Lₓ·L_{y}·L_{z}=Lₓ₀·L_{y0}·L_{z0}=constant. The tensile strain was defined by the expression:

$$Tensile strain = \frac{Δ {L}_{x}}{{L}_{x 0}}$$

Tensile stress, σ, was determined by:

$$Tensile stress = F \cdot \frac{{L}_{x}}{{L}_{x 0} \cdot {L}_{y 0} \cdot {L}_{z 0}}$$

where F is the applied force.

All SAXS/WAXS experiments, including both ex-situ and in-situ measurements, were conducted at room temperature and under ambient conditions, where a Kapton window is placed between the sample environment and the vacuum beam path. To minimize background scattering contributions from the Kapton window and the beam path, a background image of the empty sample stage was acquired prior to each measurement series. The resulting background signal was subtracted from the raw data using an absorption-based correction model:

I=I_{raw}−α·I_{background}

where α represents the intensity ratio of the direct beam passing through the sample. This correction was essential to accurately isolate the scattering signal originating solely from the sample.

The SAXS/WAXS data were analyzed using SciAnalysis, a custom suite of Python-based scripts developed for batch processing of SAXS/WAXS images at NSLS-II. The scattering images were initially transformed to reciprocal space (q-space), where the scattering vector q is defined by:

$$q = \frac{4 π sin ( θ )}{λ}$$

Here, θ is the scattering angle, and is the wavelength of the incident X-ray beam. The resulting q-space data were processed to obtain circular averages, sector averages, or angular distribution profiles as required.

### Hermann Orientation Factor

$$OF = \frac{1}{2} ( 3 〈 {cos}^{2} ϕ 〉 - 1 )$$

### Example 3. Fabrication of Hierarchical Structures of GE Hydrogels

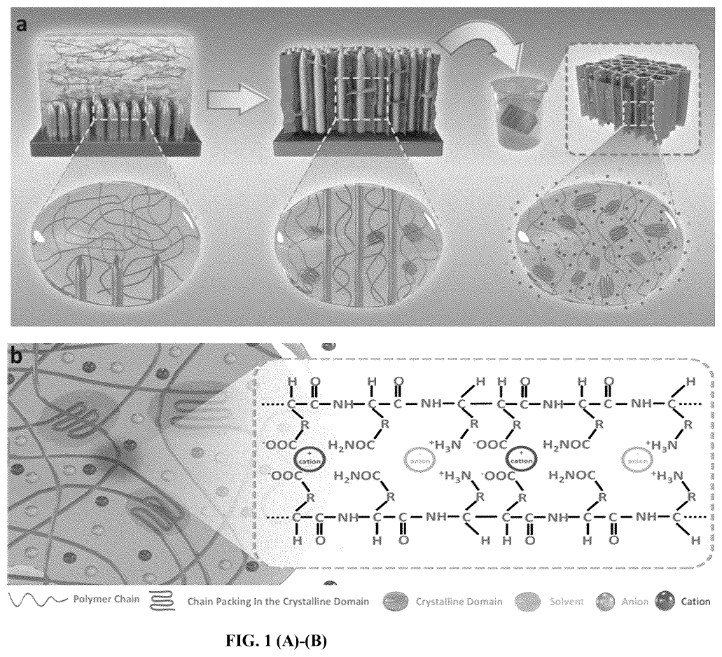

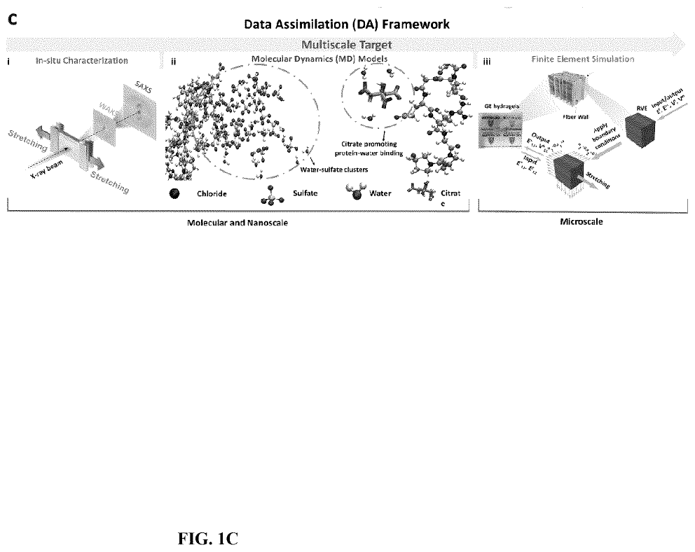

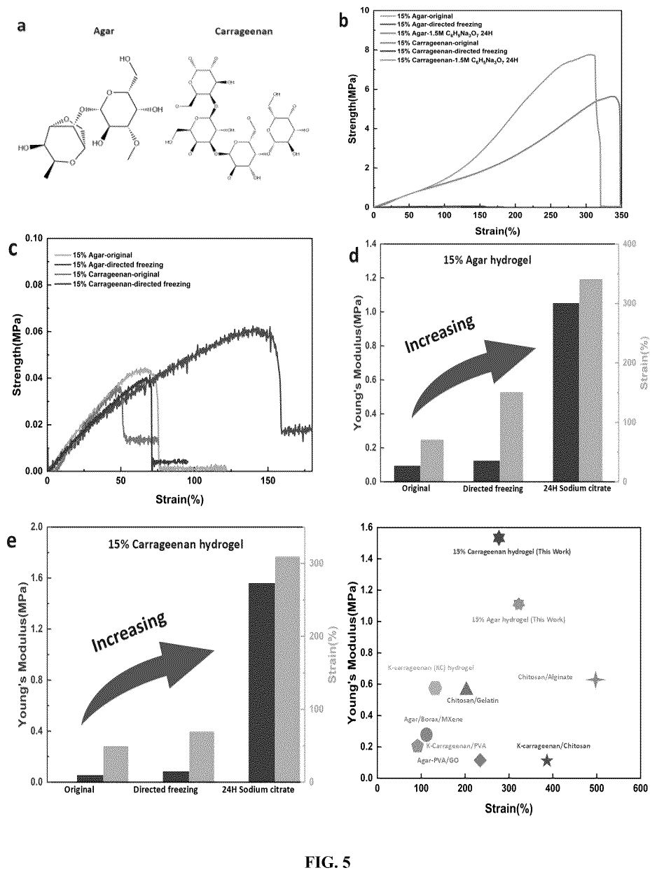

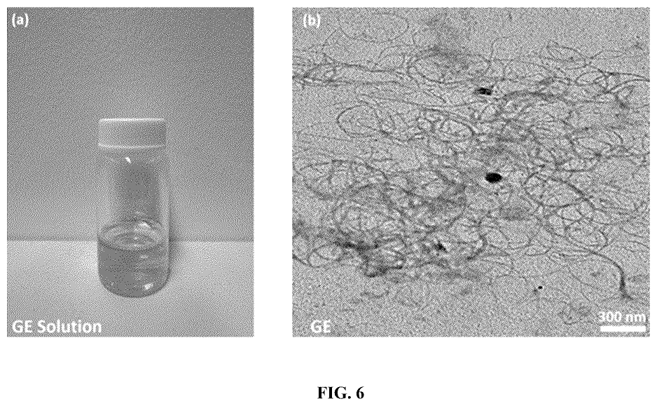

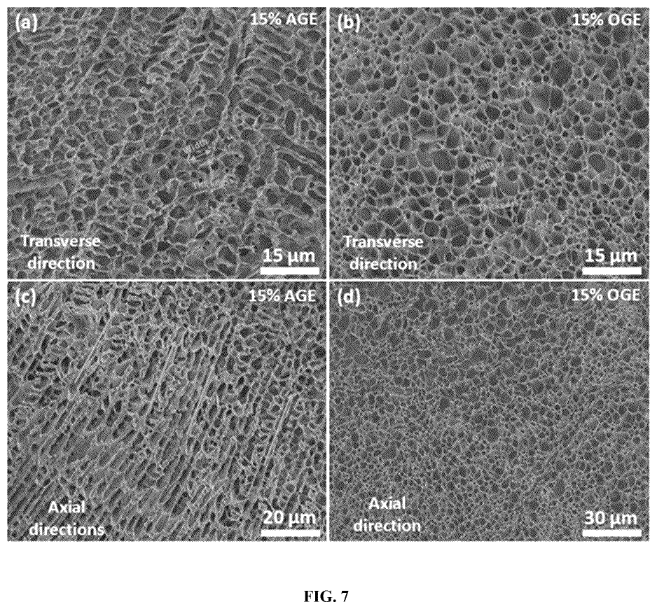

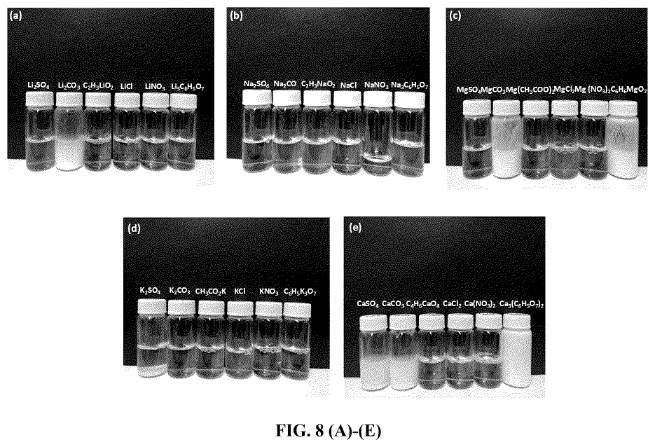

This example demonstrates a practical strategy that leverages the synergistic effects of ice separation-induced self-assembly, the spatiotemporal control of ions strategy, and hierarchical material microstructure to design anisotropic GE (AGE) hydrogels with programmable mechanical properties and cytocompatibility (FIG. 1a). The GE solution (15 wt. %) (FIG. 6a) grows icicles under a vertical temperature gradient through ultra-low temperature directional freezing. The average length of GE protein chain is around 1145 nm and the width is about 24 nm, which makes GE flexible and elastic (FIG. 6b). The steep growth of icicles induces phase separation and concentration of the GE solution, promoting the self-assembly and entanglement of GE protein chains. Concurrently, hydroxyl, amino, amide, and carboxylic acid groups on the concentrated GE protein chains establish hydrogen bonds with each other, as well as through hydrogen and oxygen atoms in peptide bonds. These hydrogen bonds play a critical role in stabilizing the secondary and tertiary structures of GE. The result is a cellular GE ice crystal microgranule network with aligned pore walls, forming AGE hydrogels with a neatly oriented structure along the axial direction (FIGS. 7a and 7c). Compared to untreated original GE (OGE) hydrogels, these hydrogels exhibit a higher degree of amorphousness and a lower degree of crystallinity (FIGS. 7b and 7d). Subsequently, GE hydrogels with widely programmable mechanical properties were achieved via ion spatiotemporal expression modulate. After ice separation-induced self-assembly, the GE ice crystal building blocks are immersed in 1.5M various metal salt solutions (FIG. 8) and stored at 5 °C until the ion spatiotemporal expression modulate was completed.

Ion spatiotemporal expression modulate the interactions of GE protein molecular chains, leading to the changes in polymer chain conformation and variation of crystallinity to make GE hydrogels show a wide range of adjustable mechanical properties (FIG. 1b). A series of GE hydrogels were fabricated; the detailed name are shown in Supplementary Tables 1.

TABLE 1
GE Hydrogel Abbreviations and Full Names Comparison Table

| Full Names | Abbreviations |
|---|---|
| Anisotropic GE-Lithium Sulfate | AGE-LS |
| Anisotropic GE-Lithium Carbonate | AGE-LKC |
| Anisotropic GE-Lithium Acetate | AGE-LA |
| Anisotropic GE-Lithium Citrate | AGE-LC |
| Anisotropic GE-Sodium Sulfate | AGE-SS |
| Anisotropic GE-Sodium Carbonate | AGE-SKC |
| Anisotropic GE-Sodium Acetate | AGE-SA |
| Anisotropic GE-Sodium Chloride | AGE-SCL |
| Anisotropic GE-Sodium Citrate | AGE-SC |
| Anisotropic GE-Magnesium Sulfate | AGE-MS |
| Anisotropic GE-Magnesium Carbonate | AGE-MKC |
| Anisotropic GE-Magnesium Acetate | AGE-MA |
| Anisotropic GE-Magnesium Citrate | AGE-MC |
| Anisotropic GE-Potassium Sulfate | AGE-PS |
| Anisotropic GE-Potassium Carbonate | AGE-PKC |
| Anisotropic GE-Potassium Acetate | AGE-PA |
| Anisotropic GE-Potassium Chloride | AGE-PCL |
| Anisotropic GE-Potassium Citrate | AGE-PC |
| Anisotropic GE-Calcium Sulfate | AGE-CS |
| Anisotropic GE-Calcium Carbonate | AGE-CKC |
| Anisotropic GE-Calcium Acetate | AGE-CA |
| Anisotropic GE-Calcium Citrate | AGE-CC |
| Original GE | OGE |
| Anisotropic GE | AGE |

GE hydrogels are treated with calcium chloride, lithium chloride, magnesium chloride, and all nitrate salts melted at room temperature. This behavior is attributed to nitrate ions, which, as weakly hydrated chaotropic ions, disrupt the hydrogen bonding and hydrophobic interactions between GE molecules. Additionally, the high ionic strength and cationic shielding effects of calcium, magnesium, and lithium ions further reduce the crosslinking density and thermal stability of the hydrogel. These hydrogels will not be included in further research.

Ex-situ and in-situ Wide-angle X-ray scattering (WAXS) and in-situ small-angle X-ray scattering (SAXS) elucidate the mechanisms driving changes in the hierarchical structure of GE hydrogels, from molecular chain spacing to nanoscale layers. These structural transformations directly impact the rigidity, flexibility, and energy dissipation capacity of the hydrogels, ultimately modulating their overall mechanical properties.

### Example 4. Characterization and Ionic Spatiotemporally Programmable Mechanism Profiling

a. Broadly Programmable Mechanical Properties

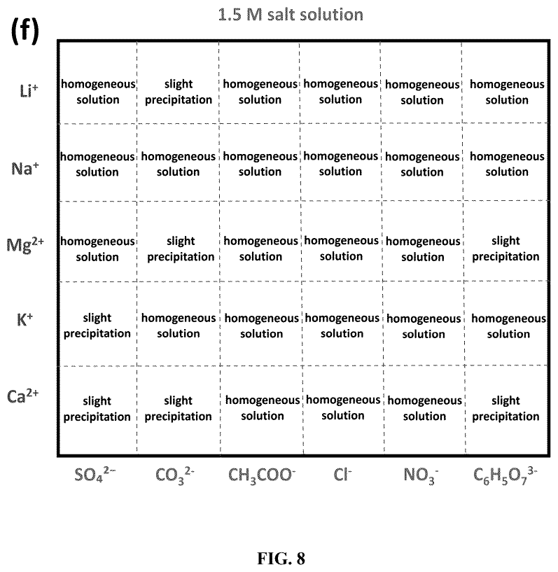

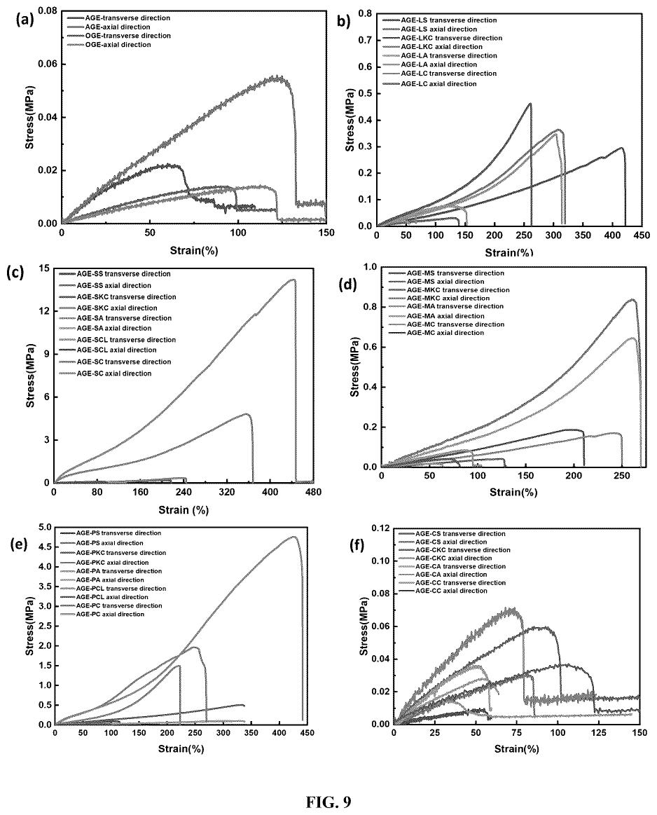

The mechanical properties of AGE hydrogels were characterized along both the oriented (axial) and non-oriented (transverse) directions using a universal testing machine, allowing for a comparative analysis of their mechanical performance. The tensile strength of AGE hydrogels increased significantly along the axial direction compared to the OGE hydrogels (FIG. 9), highlighting the role of the alignment structure in resisting damage under load. Typical stress-strain curves of AGE hydrogels, programmed via ice separation-induced self-assembly and ionic spatiotemporal modulation, are illustrated in FIG. 9. The AGE-SC hydrogel exhibited the highest ultimate stress (14.25 MPa) and strain (450%) along the axial direction, whereas the AGE-PCL hydrogel displayed the lowest ultimate stress (6.79 kPa) and strain (100%) along the transverse direction. The tensile strength of the strongest hydrogel was approximately 2,000 times greater than that of the weakest hydrogel. AGE hydrogels demonstrate broadly programmable mechanical properties, with tensile strength ranging from 6±1 kPa to 14.25±1 MPa, elongation at break varying from 50% to 450%, and Young's modulus spanning 9.7±0.2 kPa to 3.595±0.120 MPa (Table 2).

TABLE 2
GE hydrogels Tensile Strength and Young's Modules

| | | | Axis | Axis |
|---|---|---|---|---|
| | Transverse | Transverse | direction | direction |
| | direction | direction | Tensile | Young's |
| | Tensile | Young's | Strength | Modules |
| Sample | Strength (MPa) | Modules (MPa) | (MPa) | (MPa) |
| AGE-LS | 0.295 | 0.030 | 0.365 | 0.084 |
| AGE-LKC | 0.031 | 0.035 | 0.058 | 0.112 |
| AGE-LA | 0.021 | 0.055 | 0.077 | 0.094 |
| AGE-LC | 0.347 | 0.055 | 0.462 | 0.105 |
| AGE-SS | 0.049 | 0.069 | 0.098 | 0.127 |
| AGE-SKC | 0.194 | 0.031 | 0.341 | 0.091 |
| AGE-SA | 0.049 | 0.039 | 0.075 | 0.099 |
| AGE-SCL | 0.008 | 0.026 | 0.062 | 0.081 |
| AGE-SC | 4.84 | 2.407 | 14.25 | 3.595 |
| AGE-MS | 0.183 | 0.099 | 0.831 | 0.198 |
| AGE-MKC | 0.041 | 0.016 | 0.172 | 0.071 |
| AGE-MA | 0.084 | 0.133 | 0.643 | 0.152 |
| AGE-MC | 0.017 | 0.058 | 0.043 | 0.077 |
| AGE-PS | 0.087 | 0.098 | 0.095 | 0.160 |
| AGE-PKC | 0.513 | 0.114 | 1.492 | 0.330 |
| AGE-PA | 0.101 | 0.022 | 0.107 | 0.031 |
| AGE-PCL | 0.006 | 0.009 | 0.007 | 0.017 |
| AGE-PC | 1.971 | 0.842 | 4.764 | 1.175 |
| AGE-CS | 0.008 | 0.025 | 0.029 | 0.052 |
| AGE-CKC | 0.059 | 0.111 | 0.069 | 0.157 |
| AGE-CA | 0.028 | 0.068 | 0.035 | 0.084 |
| AGE-CC | 0.014 | 0.061 | 0.036 | 0.066 |
| OGE | 0.013 | 0.054 | 0.013 | 0.061 |
| AGE | 0.022 | 0.014 | 0.054 | 0.021 |

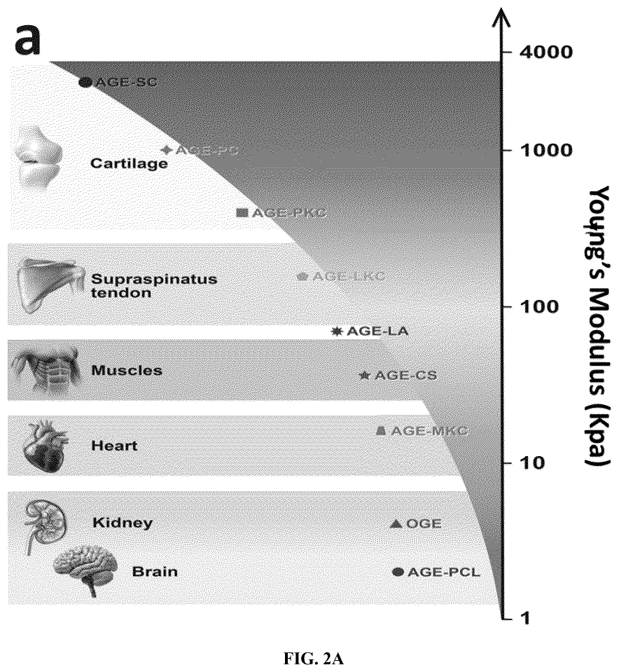

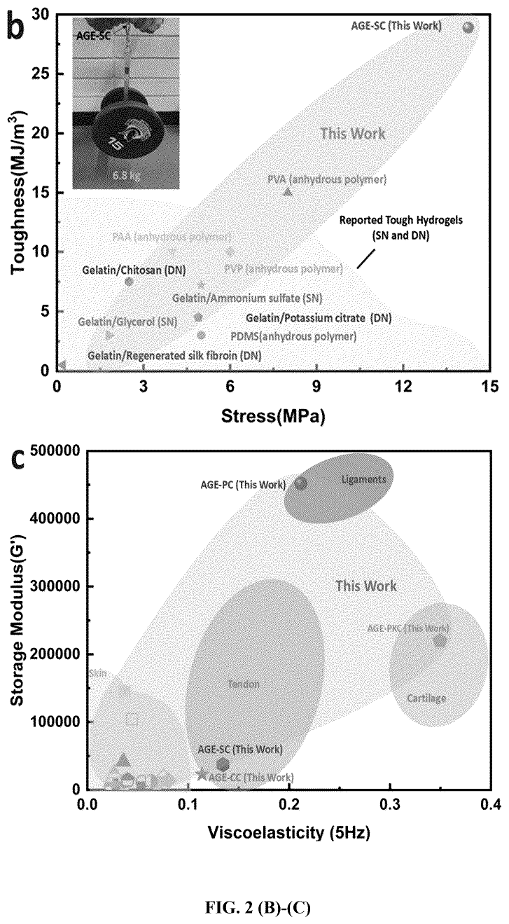

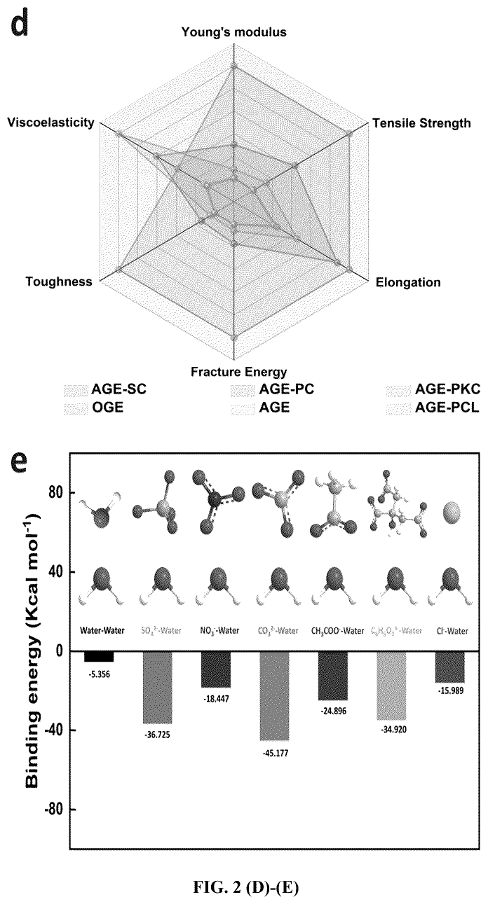

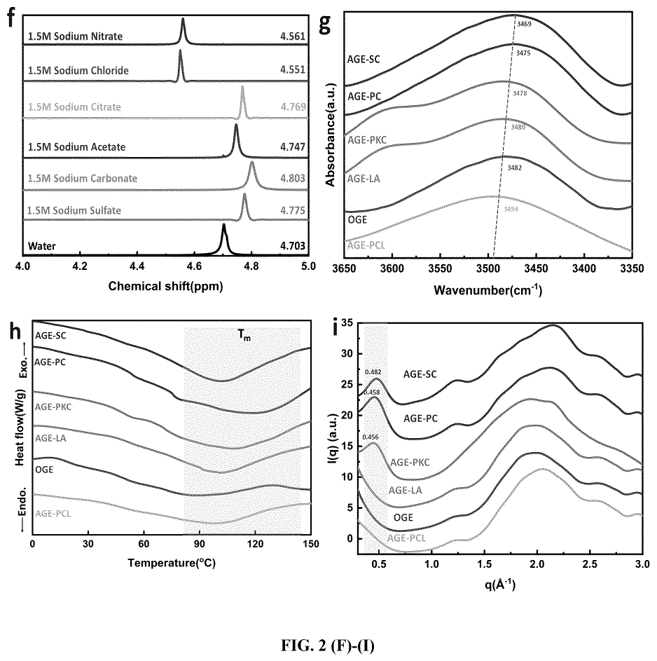

This wide range of Young's modulus encompasses the mechanical properties of various human tissues, from soft tissues like the brain, kidney, heart, and muscle to harder tissues such as tendons and cartilage (FIG. 2A).

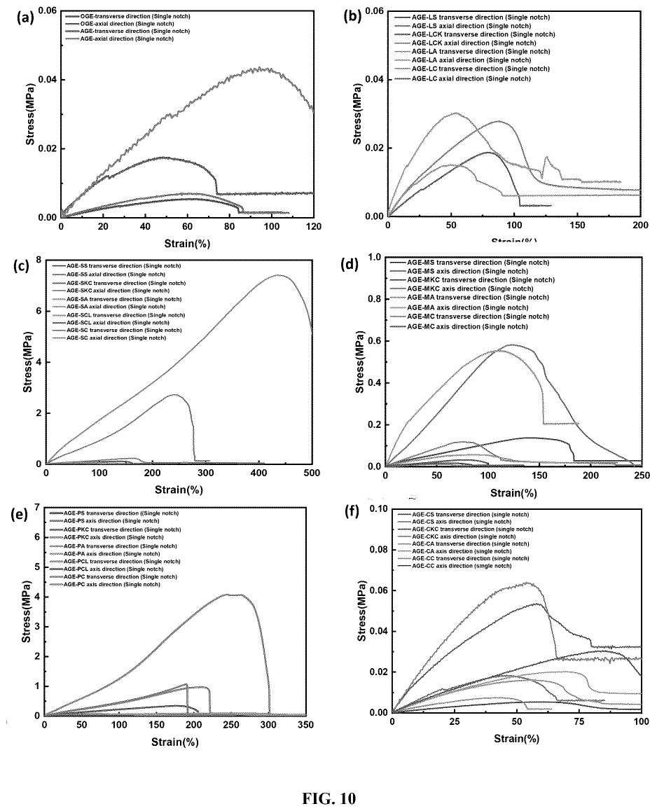

Tensile fracture tests of both notched and unnotched AGE hydrogels were performed to investigate their fracture behavior (FIGS. 9 and 10). The AGE hydrogel exhibits better defect resistance in the axial direction compared to the transverse direction, which is consistent with the results from unnotched tensile tests. Table 3 compares the toughness and fracture energy of GE hydrogels. AGE-SC hydrogel achieved a toughness of 28.89 MJ·m⁻³ and a fracture energy of 413304.7 J·m⁻² in the longitudinal direction, which were 5460 times higher than the toughness of the OGE hydrogels and 2930 times higher than the fracture energy of the OGE hydrogels demonstrating excellent defect resistance.

TABLE 3
GE hydrogels fracture toughness and fracture energy (single notch)

| | Transverse | Axis | | Axis |
|---|---|---|---|---|
| | direction | direction | Transverse | direction |
| | Fracture | Fracture | direction Fracture | Fracture |
| | Toughness | Toughness | energy | energy |
| Sample | (MJ m⁻³) | (MJ m⁻³) | (J m⁻²) | (J m⁻²) |
| AGE-LS | 0.183 | 0.205 | 3625.2 | 4061.1 |
| AGE-LKC | 0.0104 | 0.0181 | 275.6 | 479.7 |
| AGE-LA | 0.0051 | 0.013 | 149.4 | 380.9 |
| AGE-LC | 0.109 | 0.131 | 2343.5 | 2816.5 |
| AGE-SS | 0.013 | 0.025 | 372.8 | 714.6 |
| AGE-SKC | 0.07 | 0.12 | 1647.5 | 2658.4 |
| AGE-SA | 0.011 | 0.034 | 312.6 | 897.5 |
| AGE-SCL | 0.0037 | 0.017 | 101.6 | 485.8 |
| AGE-SC | 3.43 | 28.89 | 66986.1 | 413304.7 |
| AGE-MS | 0.148 | 0.191 | 3357.2 | 4567.5 |
| AGE-MKC | 0.0088 | 0.0198 | 252.4 | 558.5 |
| AGE-MA | 0.043 | 0.107 | 1086.2 | 2758.2 |
| AGE-MC | 0.0060 | 0.0165 | 186.4 | 476.3 |
| AGE-PS | 0.0251 | 0.0308 | 678.9 | 861.2 |
| AGE-PKC | 0.207 | 0.809 | 4478.1 | 16599.2 |
| AGE-PA | 0.108 | 0.121 | 2136.1 | 2397.9 |
| AGE-PCL | 0.00127 | 0.0032 | 33.9 | 89.7 |
| AGE-PC | 1.29 | 2.51 | 27310.1 | 48369.4 |
| AGE-CS | 0.00219 | 0.00437 | 65.8 | 132.1 |
| AGE-CKC | 0.0138 | 0.0147 | 385.1 | 466.9 |
| AGE-CA | 0.00738 | 0.00977 | 209.6 | 278.4 |
| AGE-CC | 0.00965 | 0.0156 | 296.3 | 409.5 |
| OGE | 0.00330 | 0.00336 | 92.6 | 96.2 |
| AGE | 0.00590 | 0.0208 | 176.2 | 556.4 |

At a water content of over 70% hydrogels, AGE-SC hydrogel surpassed all other reported GE-based hydrogels whether single or dual network in terms of tensile strength and toughness, also well above those of water-free polymers such as polydimethylsiloxane (PDMS), Polyacrylic acid (PAA), Polyvinylpyrrolidone (PVP), and Polyvinyl alcohol (PVA) (FIG. 2b). The AGE-SC hydrogel can easily lift 15 pounds dumbbells never broken.

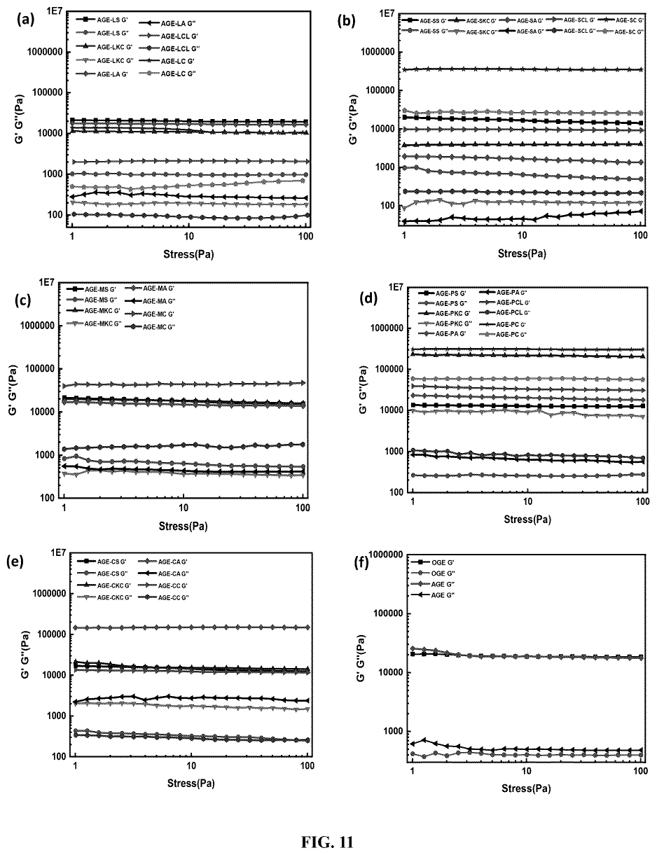

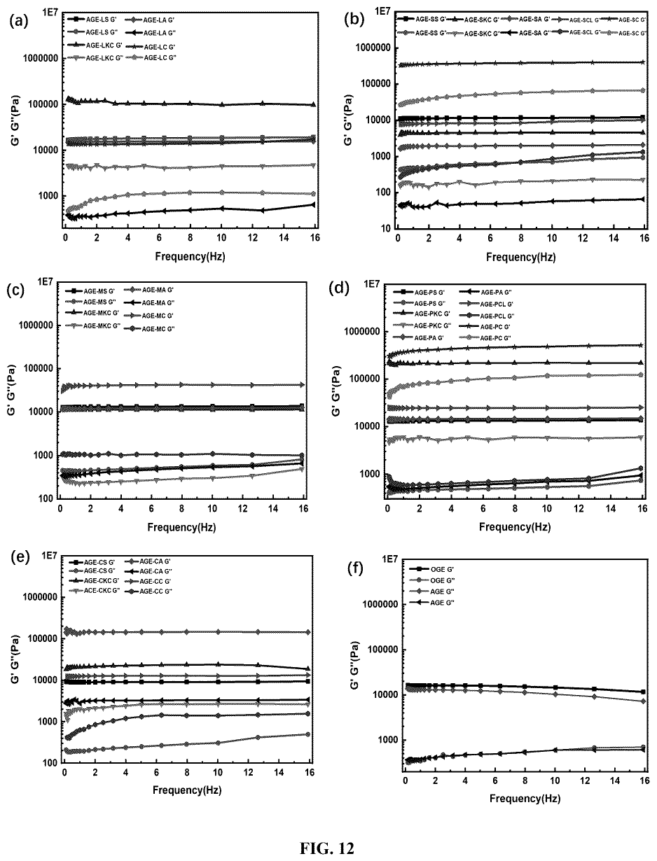

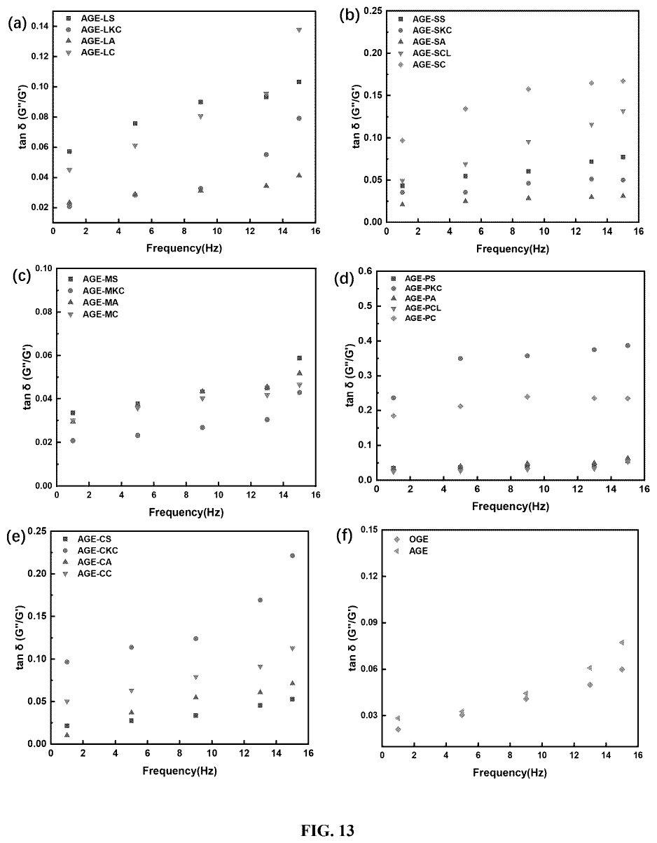

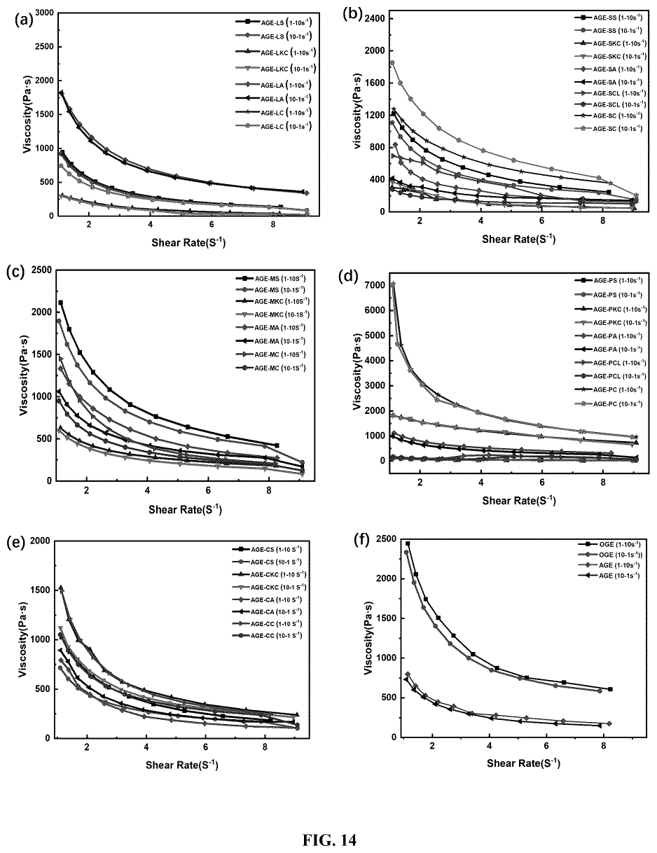

Rheological testing offers deeper insights into the response of GE hydrogel to various stresses and strains and evaluates the viscoelastic properties of hydrogels. Three models were employed to study the rheological properties of the prepared GE hydrogels (FIGS. 11-13). Dynamic stress amplitudes sweeps patterns at a fixed frequency of 1 Hz reveal the linear viscoelastic region of the hydrogel (FIG. 11). Interestingly, all hydrogels' storage modulus (elastic modulus, G′) values are much higher than the loss modulus (viscous modulus, G″), indicating that all samples can maintain the ideal elastic state. Compared with the elastic modulus of the original GE hydrogel as 20600 Pa, the elastic modulus G′ of AGE-SC hydrogel increased to 36246 Pa, which showed that the ice separation-induced self-assembly and ionic spatiotemporal modulate enhanced the rigidity of the hydrogel carrier. Frequency sweep mode was performed on all GE hydrogels under constant stress of 1 Pa (FIG. 12). All G′ and G″ modulus of the gel are essentially frequency independent. At the same time, G′ is one magnitude higher than G″. The rheological behavior shows typical solid-state behaviour with oscillation frequencies ranging from 0.159 to 15.9 Hz. Due to the reduced rearrangement capacity, diminished energy dissipation, enhanced intermolecular interactions, and network entanglement of GE protein chains, the viscoelasticity of GE hydrogels increases with increasing frequency (FIG. 13). The viscoelasticity of GE hydrogels can perfectly match tissues such as skin, tendon [AGE-SC hydrogel and AGE-CKC (anisotropic 15% GE-1.5M Calcium carbonate) hydrogel], ligament (AGE-PC (anisotropic 15% GE-1.5M Potassium citrate hydrogel), and cartilage (AGE-PKC (anisotropic 15% GE-1.5M Potassium carbonate hydrogel) (FIG. 2c). Among them, the AGE-SC hydrogel exhibited superb tensile properties, high strength, outstanding ductility, and excellent toughness (FIG. 2d). GE hydrogels are formed through reversible non-covalent bonds, which gives the hydrogels excellent shear-responsive flow behavior. Continuous flow and strain measurements evaluated the shear thinning and self-healing properties of GE hydrogels. GE hydrogels show different initial viscosities when no shear force is applied, due to various interaction between GE protein molecular chain (FIG. 14). The viscosity of the all GE hydrogel decreased with an increasing shear rate from 0.1 s⁻¹ to 10 s⁻¹. In contrast, in the immediate reverse cycle, the viscosity returned almost to the initial state, indicating GE hydrogels have excellent shear thinning behaviour.

b. Characterization and Toughening Mechanism

DFT characterization was first performed to elaborately investigate the binding energy of different anions with water. As FIG. 2e illustrated, the binding energies of all the chaotropic anions including NO₃⁻ and Cl⁻ are lower than the typical kosmotropic SO₄²⁻, CO₃²⁻, CH₃COO⁻, and C₆H₅O₇³⁻, which is in consistency with the Hofmeister series. In general, anions can break the hydrogen bonding (HB) of water by forming the corresponding anion-H bonding through their charges, and the lower binding energy usually reveals relatively weakened bonding forming. The interruptions of the anions on the HB of water molecules are then characterized by the ¹H NMR spectra in FIG. 2f, with Na⁺ as the constant cation. Notably, the ¹H peaks all shift to higher field with the presence of chaotropic anions like NO₃⁻ and Cl⁻, substantiating that the HB of water molecules is weakened in the chaotropic anions, according well with the DFT results. In sharp contrast, the ¹H peak shifts to lower field in the kosmotropic SO₄²⁻, CO₃²⁻, CH₃COO⁻, and C₆H₅O₇³⁻ solution comparing with pure water, indicating the enhanced polarization and HB of the water molecules in kosmotropic anions, revealing the conspicuous difference of chaotropic and kosmotropic anions on the HB formation of water molecules.

To reveal the structural features of the hydrogel network after ionic spatiotemporal programming, flourier transform infrared (FTIR) spectroscopy was used to characterize the functional groups of fibrous hydrogels. The FTIR spectra of the GE hydrogels after ionic spatiotemporal programming are consistent with the hydrogel without ionic spatiotemporal programming (original GE hydrogel and anisotropic GE hydrogel) in terms of band shape and wavenumber (FIG. 15), which indicates that the main functional groups have not changed. By analyzing these FTIR spectra, it is evident that different metal salt solutions have a significant effect on the structure of GE hydrogels. Different salt treatments lead to changes in the position and intensity of the absorption peaks. The peak around 3300 cm⁻¹ is a stretching vibration of intramolecular hydrogen bonds (O—H). The changes indicate that the hydrogen bonding of GE hydrogels increases or decreases with the modulation of different ions, suggesting that in the presence of different ions, the GE chains either become more aggregated and crystalline or more dispersed and amorphous. A comparison of the infrared spectra of hydrogels with differing mechanical properties reveals distinct shifts in the O—H peak. The O—H peak of the AGE-PCL hydrogel, which mimics the modulus of human brain tissue, exhibits a significant blue shift compared to that of the OGE hydrogel. This blue shift indicates a weakening of hydrogen bonding within the AGE-PCL hydrogel. In contrast, the O—H peak of the AGE-SC hydrogel, which demonstrates optimal mechanical properties, shows a marked red shift. This red shift suggests an enhancement of hydrogen bonding, facilitated by the interaction with citrate. (FIG. 2g). In addition, significant changes were observed in the region of the stretching vibrations of the peak around 1600 cm⁻¹ (C═O) and the peak around 3500 cm⁻¹ (N—H). This indicates ions alter the molecular structure and internal intermolecular interactions of collagen by interacting with the carbonyl and amino groups of the collagen molecule.

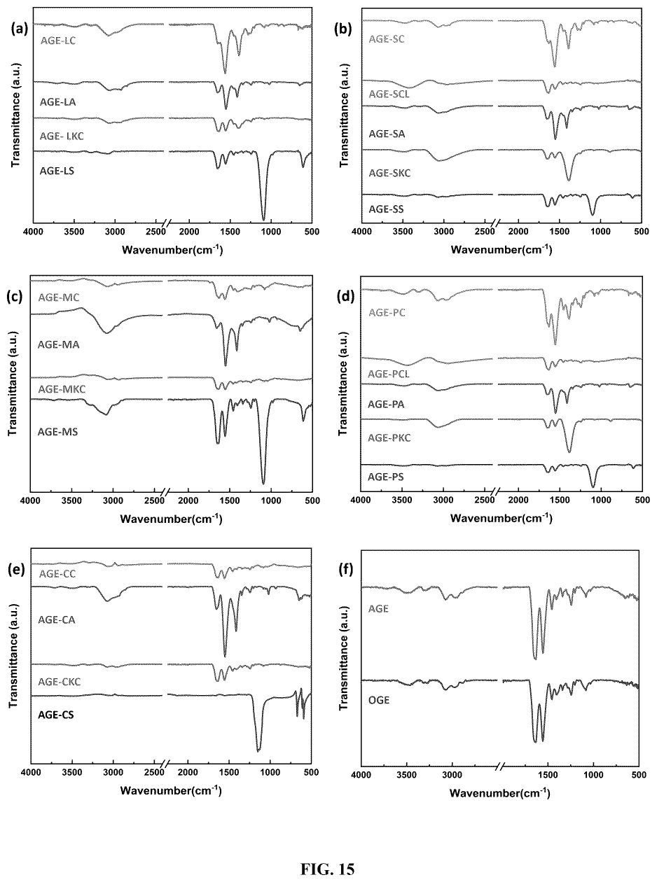

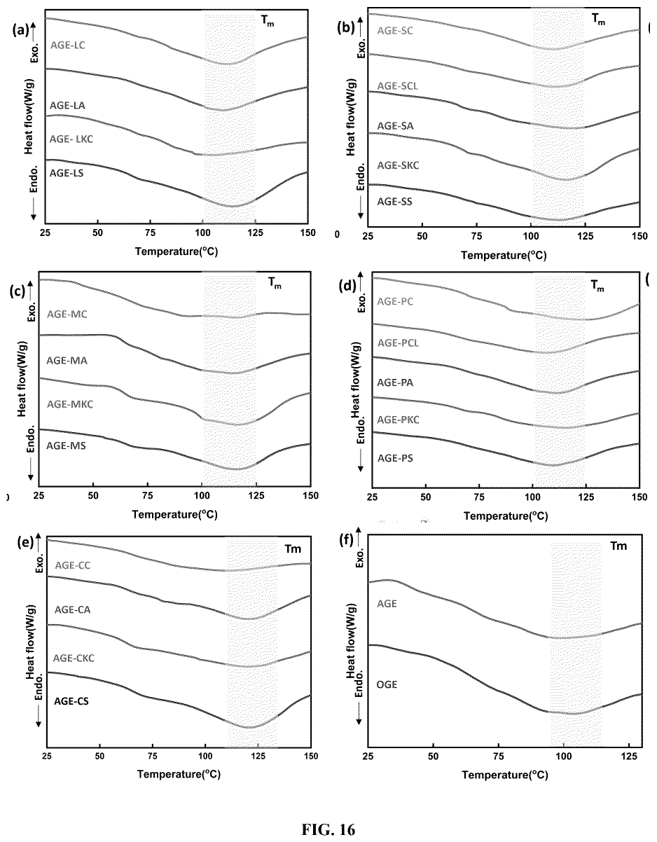

To quantify the crystalline in GE hydrogels, the GE hydrogels in the dry state were measured by differential scanning calorimetry (DSC) (FIGS. 16 and 2h). The endothermic peaks of the GE hydrogels, corresponding to melting (labelled Tₘ), are highlighted. Compared to the OGE hydrogel, the characteristic peaks in the AGE-PCL hydrogel weakened, while those in AGE-LA, AGE-PKC, AGE-PC, and AGE-SC hydrogels were progressively enhanced. This trend suggests that ionic spatiotemporal programming plays a significant role in modulating the crystallinity of hydrogels. The GE-SC hydrogel exhibited the highest crystallinity at 30.33%, a significant increase of about 3 times compared with the original hydrogel (Table 4).

TABLE 4
The crystallinity of GE hydrogels was measured by DSC. (Standard 62.5)

| Sample | ΔHc (J/g) | Xc (%) |
|---|---|---|
| AGE-LS | 14.26 | 22.82 |
| AGE-LKC | 6.74 | 10.78 |
| AGE-LA | 8.08 | 12.93 |
| AGE-LC | 15.98 | 25.57 |
| AGE-SS | 6.64 | 10.62 |
| AGE-SKC | 13.92 | 22.27 |
| AGE-SA | 9.59 | 15.34 |
| AGE-SCL | 7.49 | 12.07 |
| AGE-SC | 18.96 | 30.33 |
| AGE-MS | 10.35 | 16.56 |
| AGE-MKC | 7.35 | 11.84 |
| AGE-MA | 14.66 | 23.46 |
| AGE-MC | 7.11 | 11.38 |
| AGE-PS | 11.71 | 18.87 |
| AGE-PKC | 13.99 | 22.38 |
| AGE-PA | 10.43 | 16.69 |
| AGE-PCL | 7.01 | 11.22 |
| AGE-PC | 16.53 | 26.45 |
| AGE-CS | 6.86 | 10.98 |
| AGE-CKC | 7.62 | 12.19 |
| AGE-CA | 9.35 | 14.96 |
| AGE-CC | 6.43 | 10.29 |
| OGE | 6.56 | 10.58 |
| AGE | 5.78 | 9.32 |

This may be due to the enhancement of hydrogen bonding thereby promoting regular arrangement of molecular chains, lowering of nucleation energy barriers, increasing stability of the crystalline regions and thus increasing crystallinity. Additionally, the water content of the hydrogel, influenced by ionic spatiotemporal programmability, varies between 70% and 90% due to the different interactions between the hydrogel matrix and the various anions and cations, which can affect the water retention properties and overall structure of the hydrogel (Table 4).

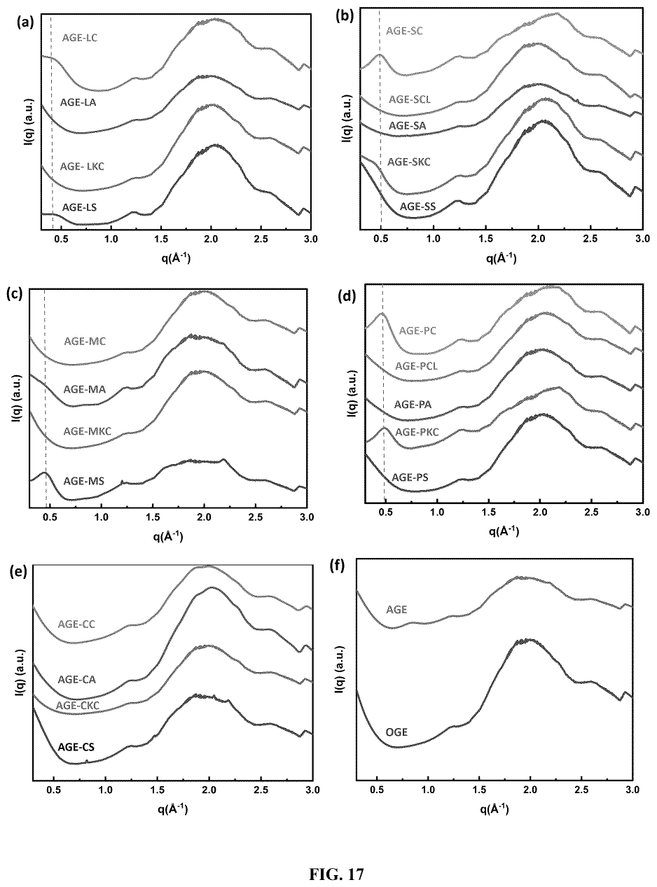

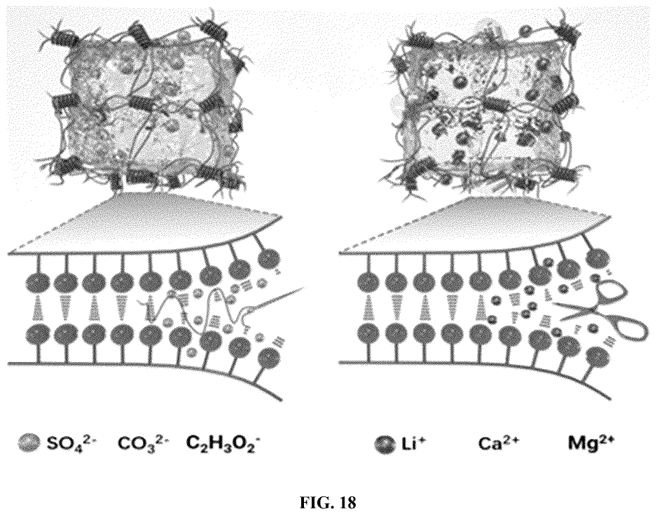

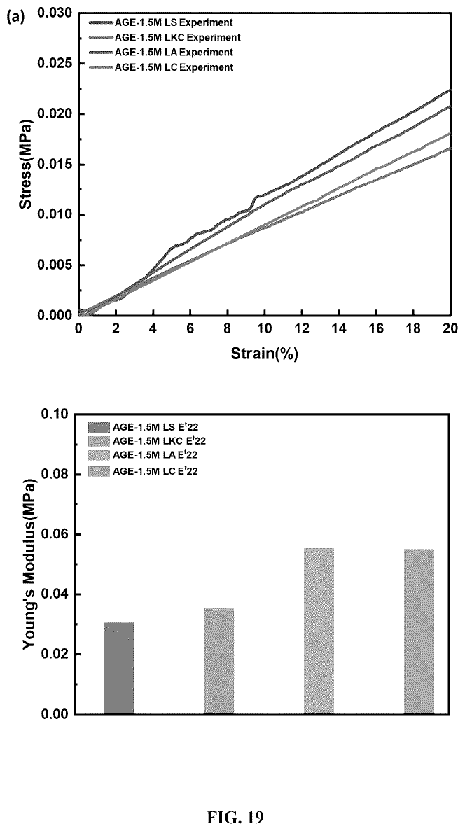

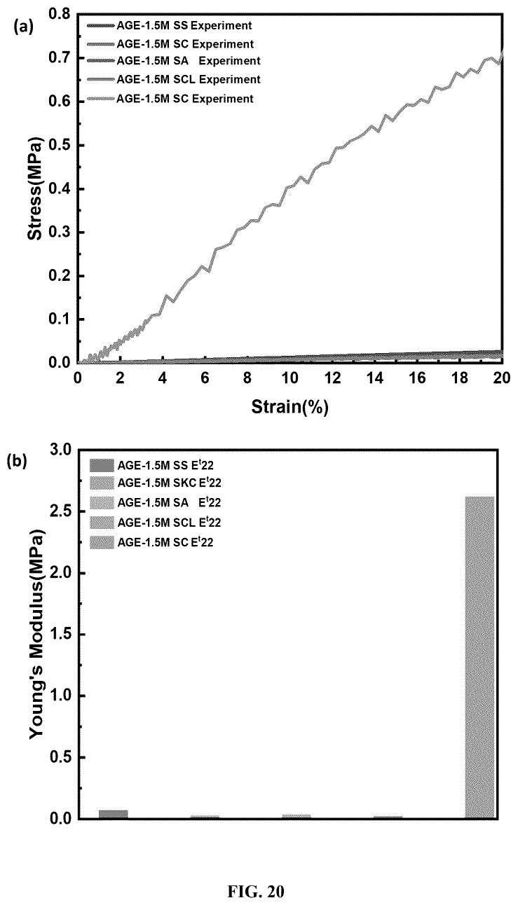

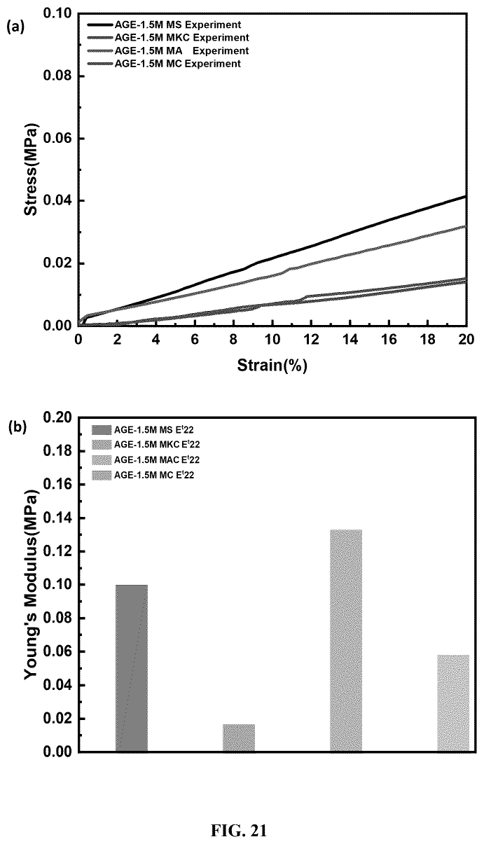

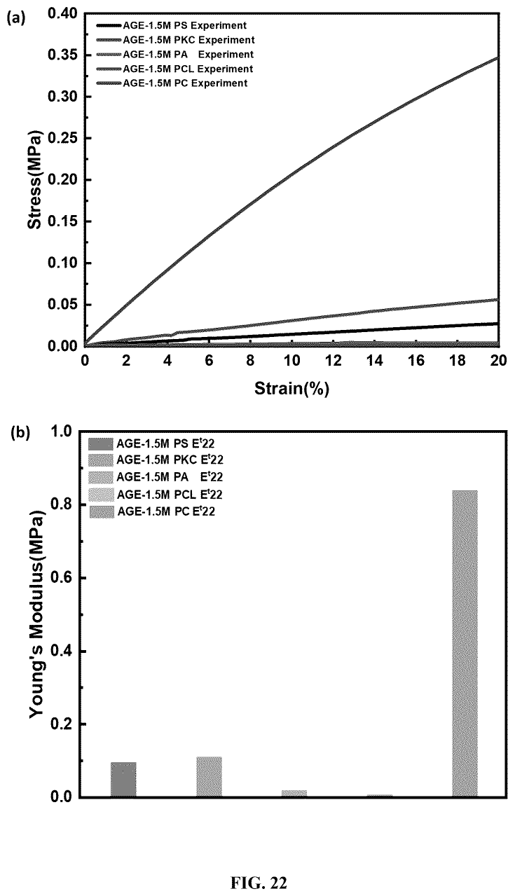

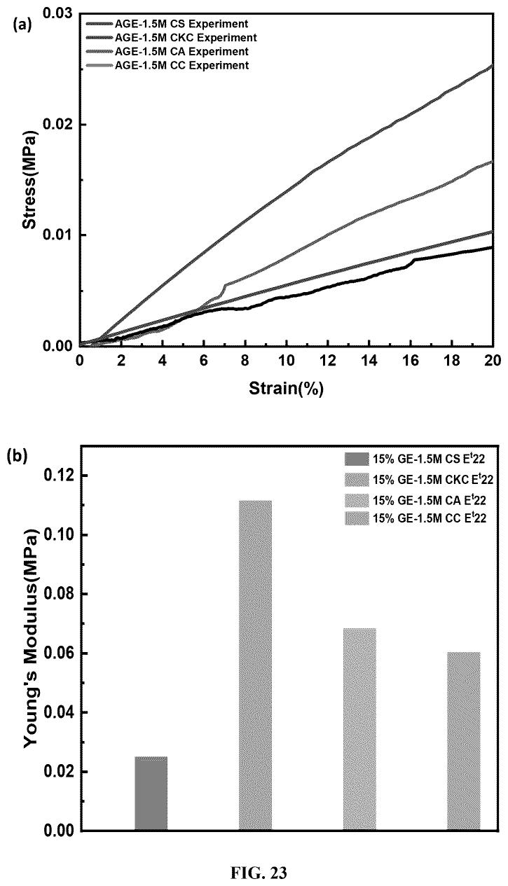

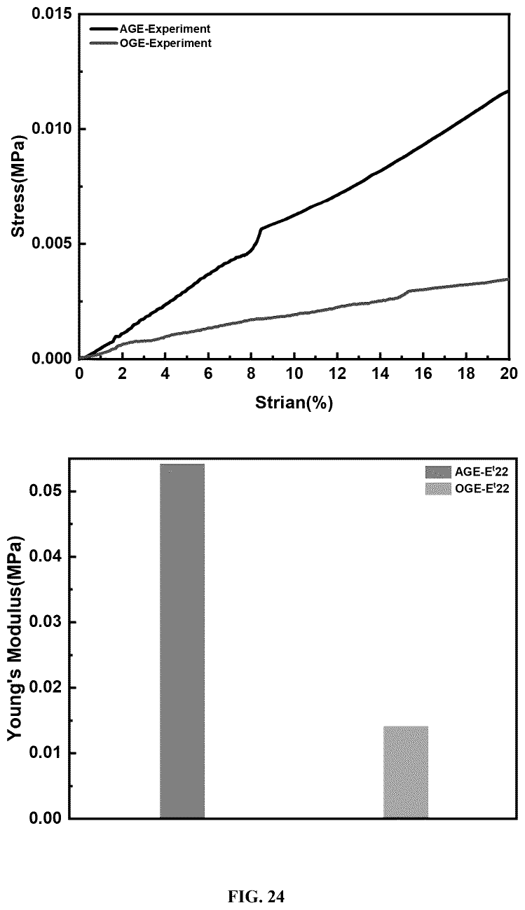

Wide-angle X-ray scattering (WAXS) was applied to further investigate and understand the mechanism of ionic spatiotemporal programming in molecular chain arrangement and crystal structure formation of GE hydrogels. FIG. 17 illustrates the ex-situ Wide-Angle X-ray Scattering (WAXS) profile of ice-separation-induced self-assembly and ionic spatiotemporal-modulation GE hydrogels. The profile features a broad peak centered at q~2.0 Å⁻¹, which is attributed to scattering from water molecules within the hydrogel matrix. Superimposed on this water-related signal is a sharper, more distinct diffraction peak at q=0.475 Å⁻¹. This peak corresponds to an average inter-chain spacing of 13.2 Å. The characteristic spacing d was calculated from the position of the scattering peak using the relation d=2π/q, where q is the scattering vector defined as q=4πλ/sin θ. This relation is commonly employed in small-angle and wide-angle X-ray scattering (SAXS/WAXS) analysis to estimate the real-space periodicity corresponding to the observed scattering features and represents the periodic arrangement of collagen molecules within the hydrogel (FIG. 2i). The inter-chain distance observed in this work is significantly larger than the values reported for pure collagen, where similar peaks have been linked to closer molecular packing. This widening of the collagen network is a direct consequence of sodium citrate insertion. Sodium citrate ions act as spacers within the collagen matrix, creating a more open molecular structure that retains sufficient periodicity to exhibit crystalline features. The introduction of sodium citrate ions also disrupts the native hydrogen-bonding network and introduces stronger ionic interactions due to its kosmotropic effect. This type of synergistic effect creates a balance between chain flexibility and crystalline order, enabling the hydrogel to exhibit superior toughness without compromising structural integrity.

### Example 5. In-Situ WAXS and SAXS Characterization for AGE-SC Hydrogels

a. Nanostructures of AGE-SC Hydrogels

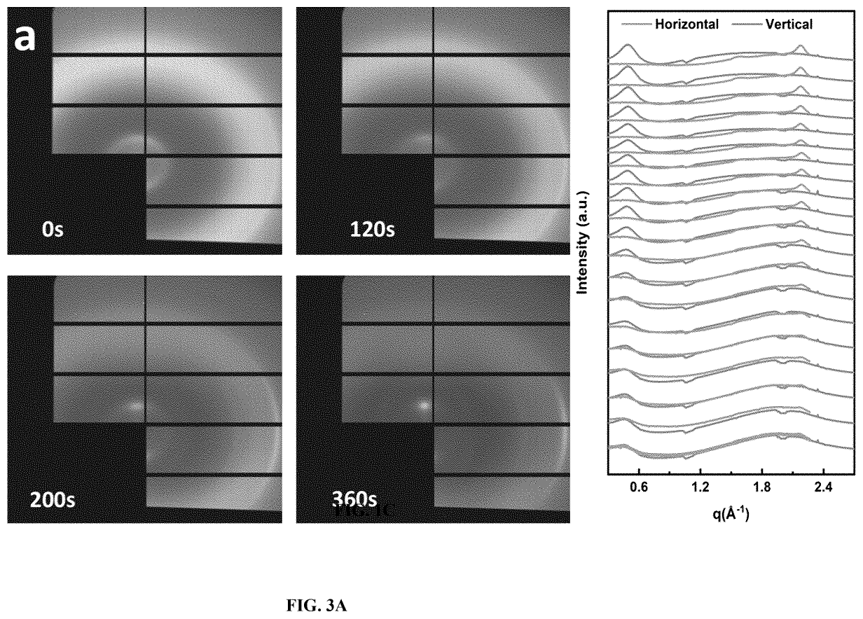

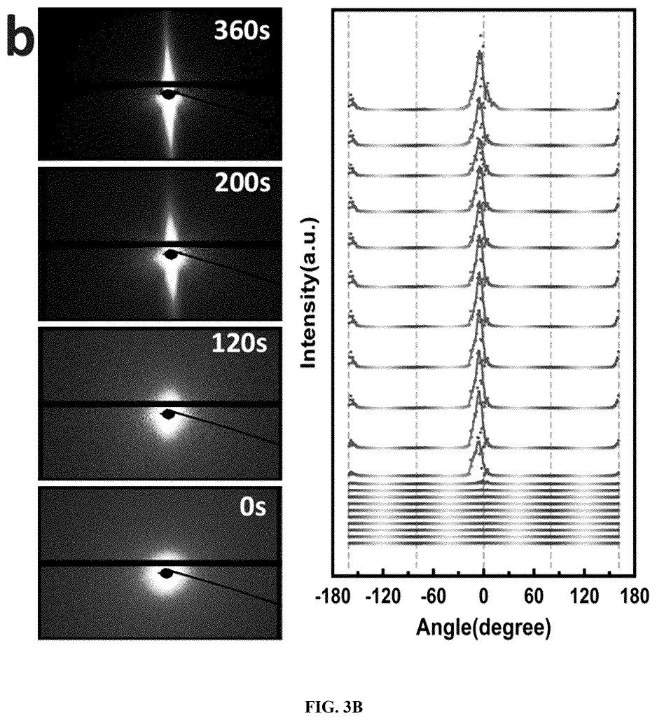

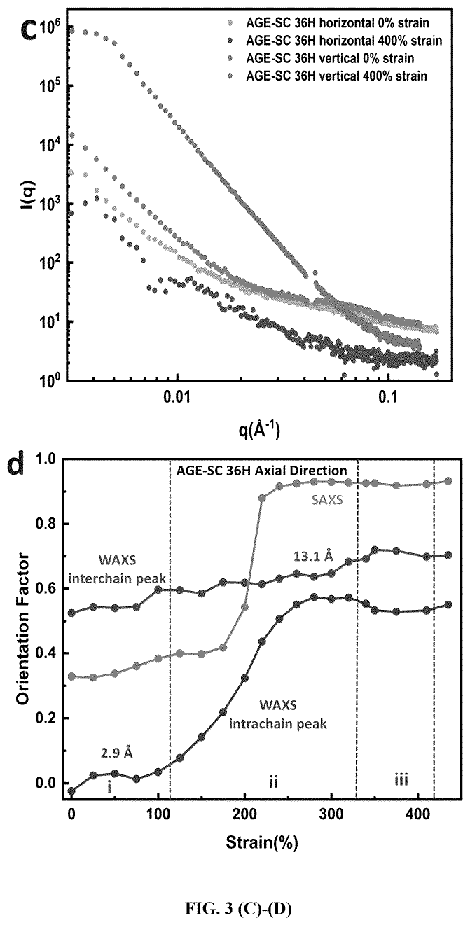

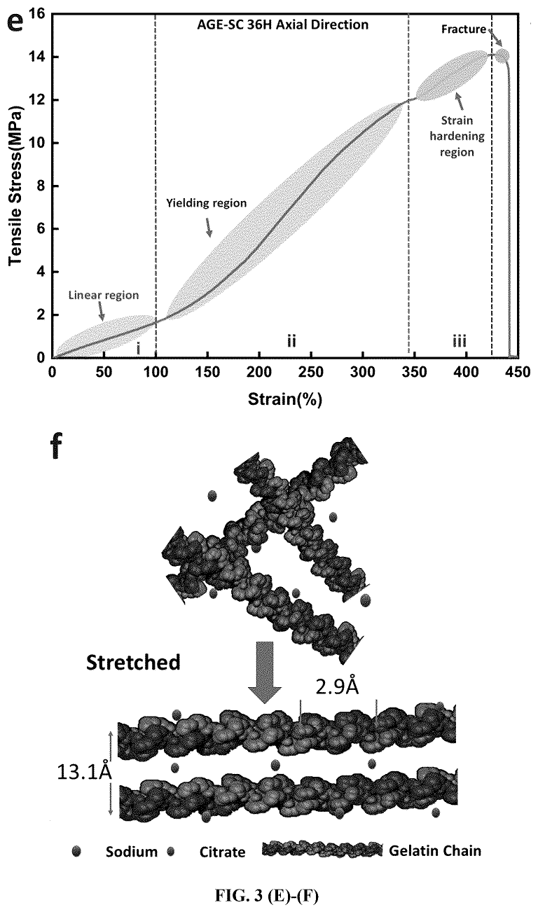

In-situ tensile WAXS/SAXS testing provides insights into the multi-scale structural and mechanical mechanisms of AGE-SC hydrogels. FIG. 3a highlights the dynamic structural evolution of AGE-SC 36H hydrogels under in-situ tensile deformation, as revealed by wide-angle x-ray scattering (WAXS). Before the application of strain, the diffraction peak at q=0.475 Å⁻¹ exhibits noticeable anisotropy in the 2D scattering pattern. The intensity is stronger along the vertical axis compared to the horizontal, indicating a preferred molecular ordering structure perpendicular to the stretching direction. This initial anisotropy reflects the pre-alignment of collagen molecules during anisotropic processing and treatment.

As strain is applied, the peak at q=0.475 Å⁻¹ transforms significantly, narrowing into a sharp diffraction spot along the vertical axis. This evolution signals the progressive alignment of collagen molecules along the stretching direction, culminating in the formation of a fringe-like structure. Simultaneously, the slight shift of the peak to q=0.500 Å⁻¹ indicates a reduction in the average collagen chain-to-chain distance to approximately 12.6 Å. This compression reflects a densification of the molecular arrangement under tensile load, where inter-chain forces and reduced steric hindrance drive the system toward a more ordered configuration. This structural alignment and compaction under tension demonstrate the role of molecular architecture in governing the hydrogel's mechanical behavior. The formation of ordered structures at the molecular scale likely enhances the material's ability to bear and distribute stress, contributing to its remarkable toughness.

Another notable feature of the in-situ WAXS data is the emergence of a secondary peak at q=2.18 Å⁻¹ as strain increases. This peak, corresponding to a periodic distance of approximately 2.9 Å, becomes evident only under significant deformation. Unlike the primary chain-to-chain diffraction peak, this secondary peak is oriented along the stretching direction and forms an arc-like pattern with an angular divergence of ±20°. This orientation and spacing are characteristic of the periodic step-to-step distance within the triple-helix structure of collagen molecules. The strain-induced appearance of this peak indicates that tensile forces promote the reorganization of molecular structures within individual collagen triple helices, suggesting a force-driven amplification of intramolecular order. At extreme deformation, the WAXS profiles reveal even higher-order peaks (FIG. 3a), indicative of a transition to a force-induced highly crystalline phase.

While WAXS provides critical insights into the crystalline and molecular-scale structure of hydrogels, small-angle x-ray scattering captures information on the organization of collagen nanofibers over length scales ranging from a few nanometers to hundreds of nanometers, complementing the molecular-scale details revealed by WAXS. This dual approach enables a comprehensive understanding of how structural hierarchies within the hydrogel respond to mechanical deformation. FIG. 3b presents the 2D SAXS patterns captured during the in-situ tensile test, showing the progressive transformation of the hydrogel's nanostructure. Prior to deformation, the SAXS patterns reveal a slightly asymmetric ellipse-like shape, with the longer axis perpendicular to the stretching direction. This anisotropy indicates a weak but measurable pre-alignment of nanostructures within the hydrogel, likely induced by the processing and treatment. Sector cuts of the SAXS profile along the vertical and horizontal direction (FIG. 3b) provide further insight into the structural details. Prior to deformation, the SAXS profiles in the low-q region (q<0.01 Å⁻¹) follows a power-law decay with slopes of I~q⁻³ along the horizontal direction and I~q^{-3.4} along the vertical direction. This behavior suggests a polydisperse distribution of nanostructures spanning the tenth-of-nano- to submicron scale, with no dominant length scale or pronounced ordering in this region. At intermediate q values (q≈0.057 Å⁻¹, d≈11 nm), a plateau emerges in the 1D SAXS profile, consistent with the form factor of nanofibers dispersed within the hydrogel matrix, which can be further supported by the TEM imaging (FIG. 6). The TEM image of free-standing gelatin reveals a randomly distributed fiber-like morphology, with an approximate diameter of tenths of nanometers, aligning well with the SAXS analysis. The directional dependence of the plateau, with slightly higher intensity along the vertical axis, reflects a weak initial orientation preference of the nanofibers.

As tensile strain is applied, the SAXS patterns remain relatively unchanged during the initial stages of stretching (FIG. 3b), indicating minimal reorganization of the structures at the nanofiber scale. However, after approximately 120 seconds, a pronounced streak emerges along the vertical axis, signifying the onset of nanostructure alignment with the stretching direction. Between 120 and 200 seconds, this streak elongates and narrows quickly, reflecting the burst formation of highly oriented nanofiber assemblies. This behavior highlights the critical role of tensile forces in driving structural alignment at the nanoscale, which also contributes to the hydrogel's mechanical response. Beyond this point, the streak continues to elongate but behaves rather slowly, suggesting sustained alignment and densification of nanostructures under increasing strain until the hydrogel ultimately fails. The power-law behavior observed in the low-q region of the SAXS profiles also tells difference at the extreme deformation. While the preservation of the I~q⁻³ slope in the horizontal direction during stretching suggests that the overall surface fractal-like organization (polydispersity) remains intact, the steepening to I~q^{-4.3} in the vertical direction at extreme strains implies the emergence of sharp interfaces, consistent with fibril fracture or reorganization during strain hardening. The initial state, the slopes of I~q⁻³ (horizontal) and I~q^{-3.4} (vertical) suggest a surface fractal-like organization, where the structure exhibits rough or irregular surfaces across multiple scales. These fractal characteristics indicate a hierarchical distribution of nanostructures, with the vertical direction being slightly more irregular, likely due to processing-induced anisotropy. The preservation of the I~q⁻³ slope in the horizontal direction during stretching suggests that the overall distribution of nanoscale features remains intact, despite significant structural alignment. However, the steepening to I~q^{-4.3} (consistent with Porod law, Iq4 vs q plot, see FIG. 3c) in the vertical direction at higher strains signals the emergence of sharp interfaces of the aligned nanofibers.

These transformations, revealed by in-situ SAXS/WAXS experiments, underscore the material's ability to adapt its molecular-scale alignment and nanofiber-scale packing to resist further deformation. The structural changes captured in these scattering patterns provide a chance to study the tensile behavior observed in mechanical tests with a real-time resolution. To elucidate the multiple-scale structural contributions underlying the mechanical performance of AGE-SC 36H hydrogels, Herman orientation factor (OF) analysis was performed on the diffraction peaks at q=0.475 Å⁻¹ and q=2.18 Å⁻¹ on the WAXS dataset (FIG. 3d) and the scattering streak between q=0.005~0.20 Å⁻¹ on the SAXS dataset (FIG. 3d). The OF of the inter-chain arrangement, associated with the q=0.475 Å⁻¹ peak, presents a linear increase from approximately 0.5 to 0.7 during the tensile test. This behavior aligns with the linear application of strain and reflects the progressive alignment of collagen molecules mediated by inter-chain hydrogen bonding (FIG. 3e). These bonds appear to serve as primary conduits for stress transfer and deformation accommodation, allowing the hydrogel to exhibit substantial elasticity and energy dissipation in the early stages of stretching. The linear trend suggests a direct, strain-driven enhancement of molecular orientation, reinforcing the hydrogel's macroscopic toughness. In contrast, the OF for the intra-chain alignment, linked to the q=2.18 Å⁻¹ peak, exhibits a sigmoidal growth pattern. During the initial deformation stages, the OF remains relatively low, indicating minimal alignment of the triple-helix structure. This observation suggests that the triple-helical domains play a passive role in stress accommodation early on, relying on inter-chain interactions to bear most of the tensile load. However, as strain increases and the inter-chain hydrogen bonds approach their limit of reorganization, the intra-chain structure begins to reorient. The on-set point of the sigmoidal curve correlates closely with the strain-hardening region of the stress-strain curve, a critical transition where the hydrogel exhibits increased stiffness and resistance to deformation. Like this peak, the OF of the SAXS intensity distribution also undergoes a burst increase, exhibiting a sigmoidal growth pattern. Compared to the concurrently measured tensile strain profile, the initial burst in the OF corresponds to the strain-softening phase, where inter-fiber interactions dominate and allow for the gradual reorganization of nanostructures toward the tensile axis. This structural transition aligns with the emergence of a sharp SAXS streak, reflecting the onset of nanofiber alignment. As strain progresses, the OF plateaus at a maximum value of 0.93, indicative of near-complete alignment of the nanofibers along the stretching direction. This high degree of orientation coincides with the strain-hardening phase of the stress-strain curve, where the aligned nanofibers act as load-bearing elements that resist further deformation and dissipate energy efficiently to the stronger hydrogen bond at the molecular level.

The analysis on in-situ SAXS/WAXS data with real-time resolution of the tensile underscores the distinct yet complementary roles of inter- and intra-chain interactions in the mechanical behavior of AGE-SC hydrogels. It also profile provides a quantitative relationship between mechanical behavior and structural evolution at different length scales. The revealed correlation between the OF analysis and strain propagation phenomenon indicates a synergistic mechanism in which the alignment and reinforcement of inter-chain hydrogen bonds progressively transfer stress to the nanofiber bundles and the underneath triple-helix collagen molecules. These hydrogen bonds and hydrophobic interaction helices contribute significantly to the hydrogel's mechanical resilience, providing a secondary level of structural support under extreme deformation (FIG. 3f).

### Example 6. Molecular-Scale Toughening Mechanism of GE Hydrogels

a. Strengthening while Toughening

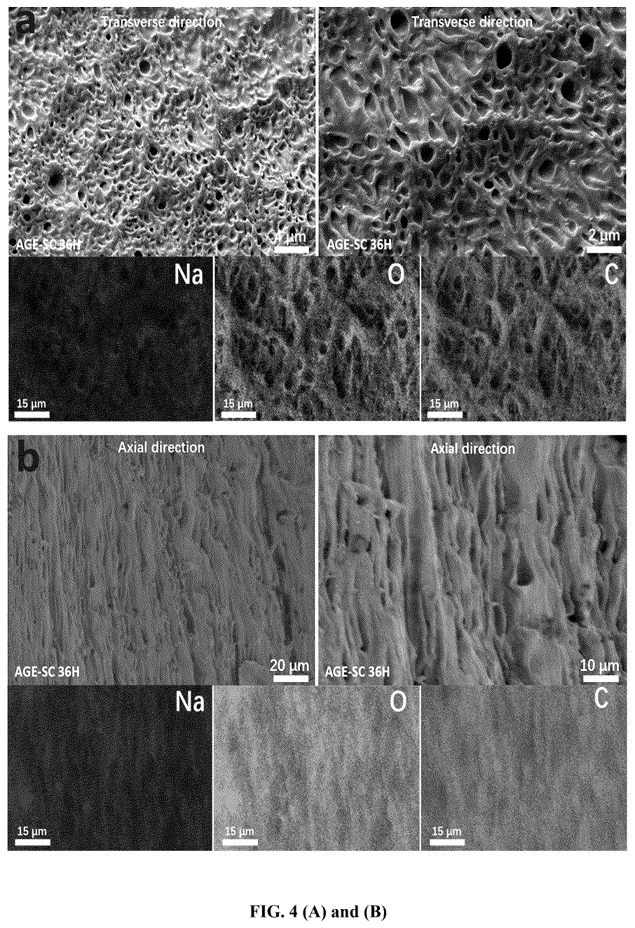

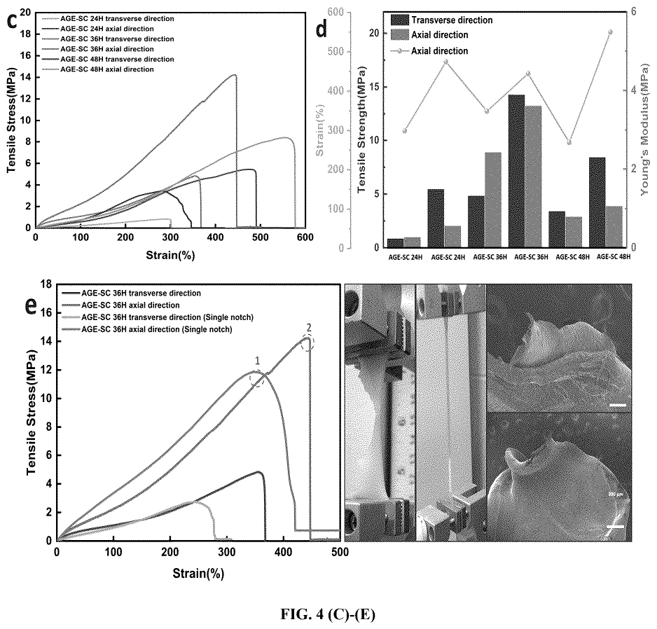

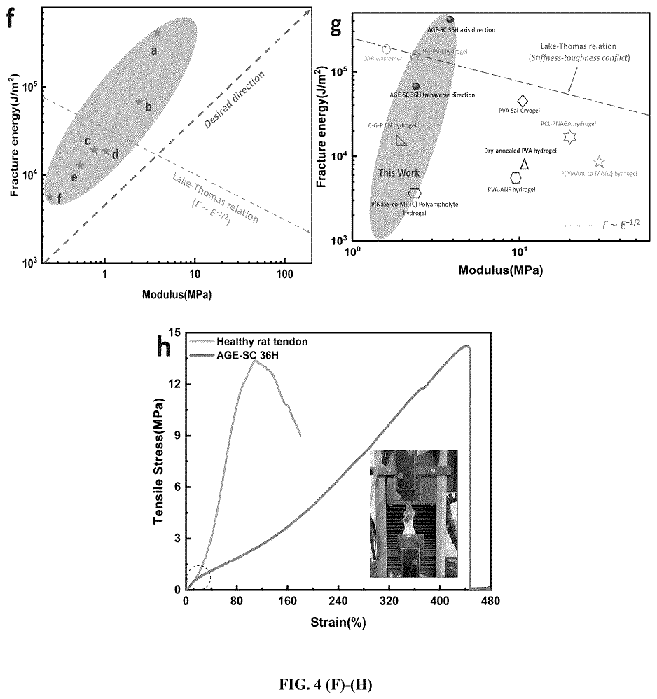

The excellent mechanical properties of the AGE-SC 36H hydrogel are attributed to its anisotropic hierarchical structure and ideal crystallization, which features uniformly distributed ultra-thick pore channel walls (FIG. 4a) and tightly stacked and highly oriented GE protein microfiber structures (FIG. 4b). EDS mapping showing a uniform distribution of Na elements indicates that sodium citrate is closely bound to the gelatin polymer chains. This indicates synergistic interactions between microfiber structures and the hydrogel matrix. These characteristics are demonstrated through microscopic and molecular characterizations. To verify the importance of ionic spatiotemporal modulation in determining the optimal time for bionic modulation of sodium citrate salt solutions, the changes in mechanical properties of anisotropic GE hydrogels were monitored at different times (24 hours, 36 hours and 48 hour) intervals in a fixed concentration of 1.5 M sodium citrate solution (FIG. 4c). Compared to the AGE-SC 24H hydrogel, the mechanical properties of the AGE-SC 36H hydrogel and AGE-SC 48H hydrogel were significantly improved. This indicates that ionic spatiotemporal modulation plays an important role in enhancing the mechanical properties of anisotropic GE hydrogels.

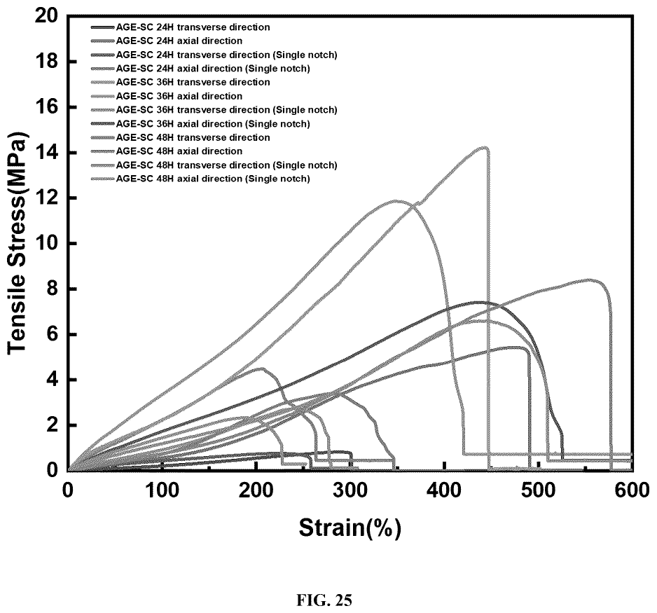

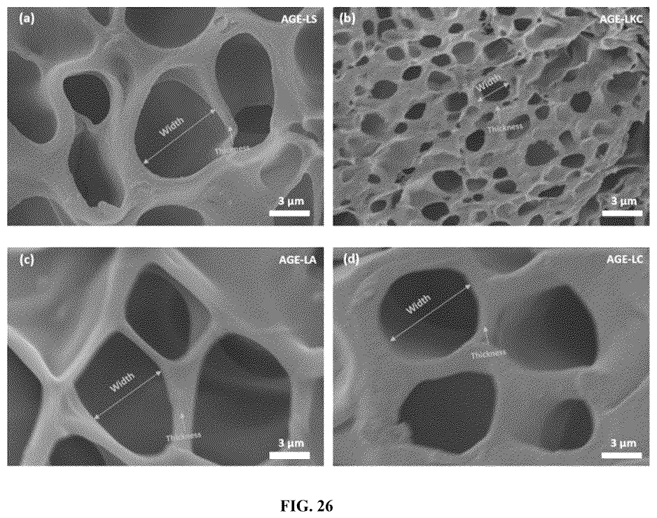

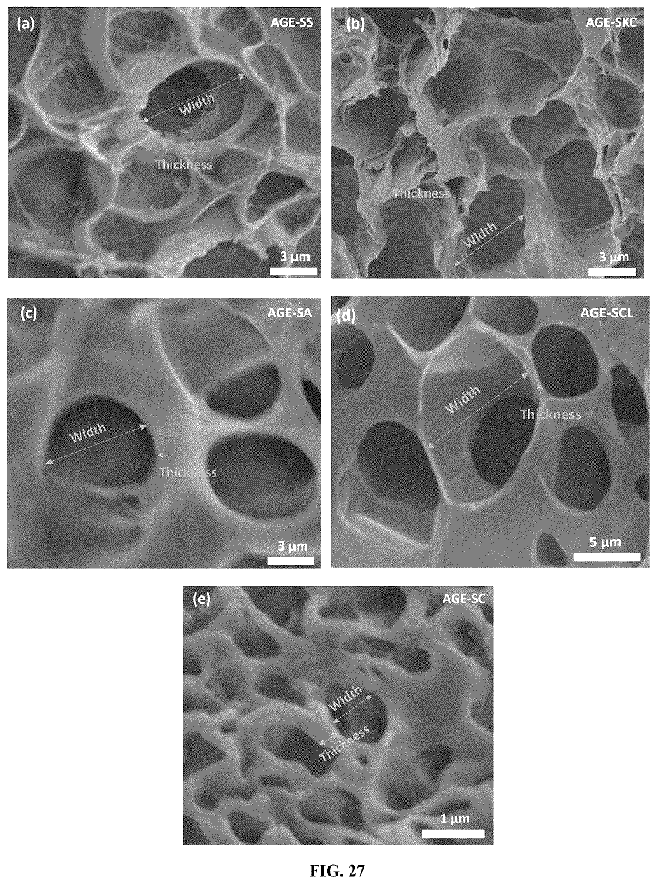

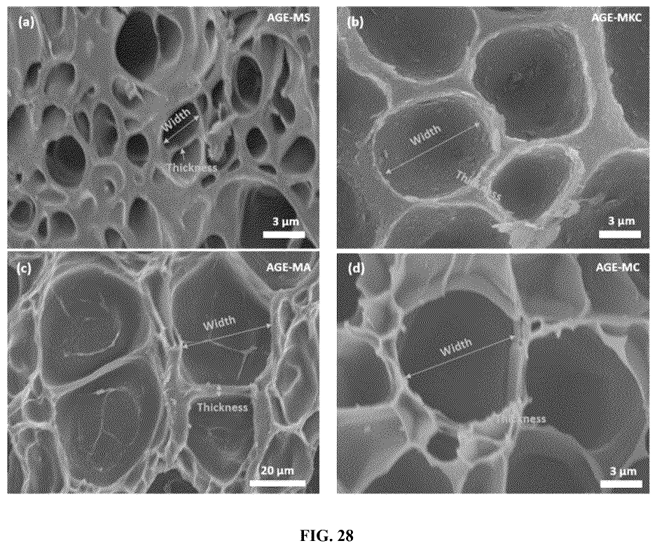

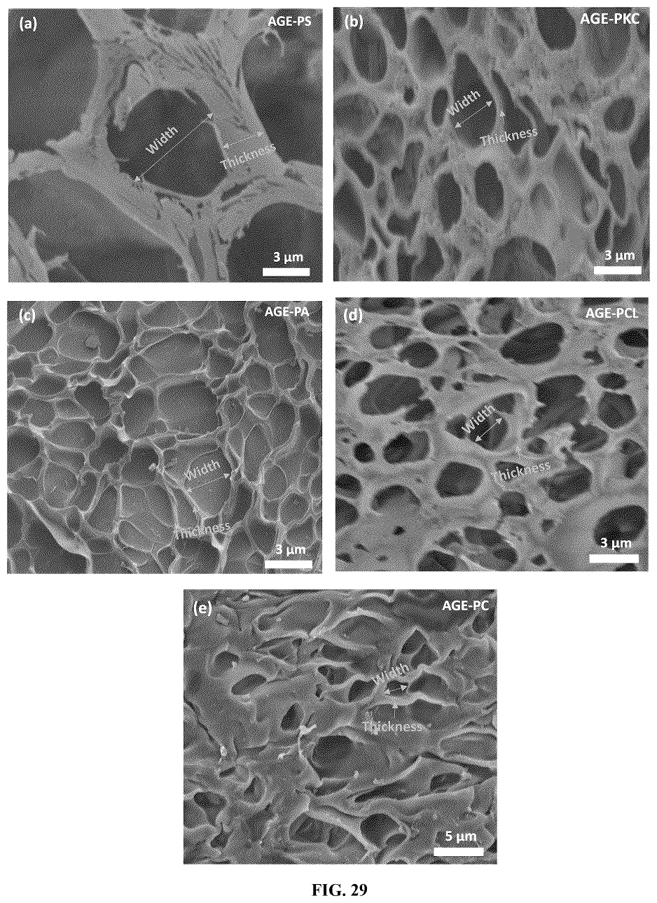

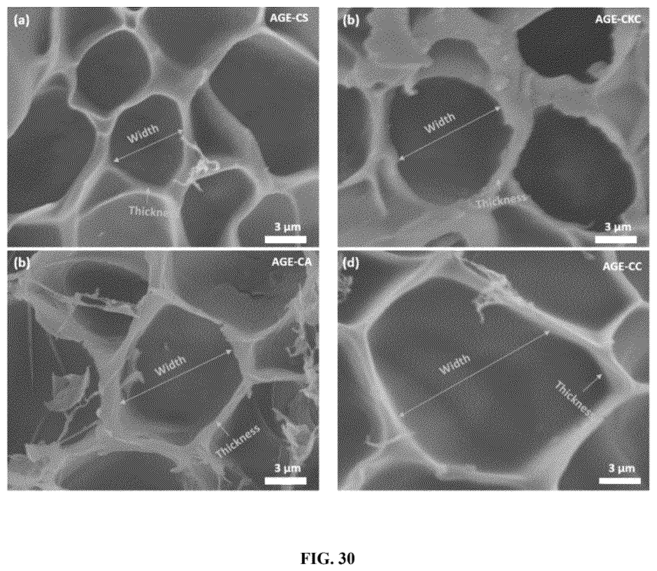

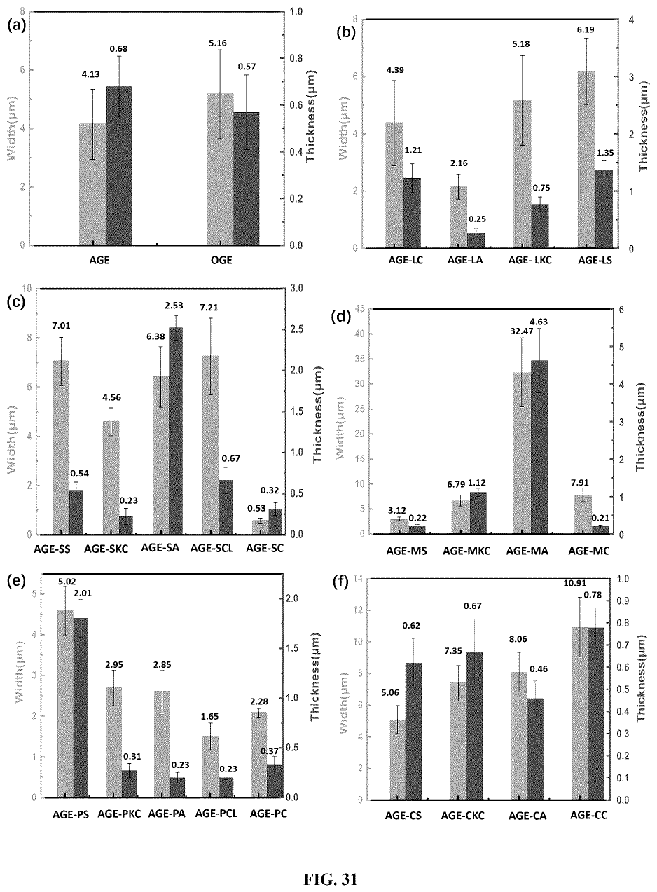

The Young's modulus and tensile strength also show a significant trend of increasing from 24 to 36 hours and then decreasing from 36 to 48 hours. Hence, the AGE-SC 36H hydrogel along the axial direction reaches a maximum tensile strength of 14.25 and Young's modulus of 3.595 MPa, respectively (FIG. 4d). The fracture mechanics test was performed on notched and unnotched AGE-SC hydrogels to study their fracture behavior (FIG. 25). Notched AGE-SC 36H hydrogel with cracks aligned perpendicular to the fibers exhibit crack-pinning ability due to the aligned fibril structure (FIG. 4e). Subsequently, the crack propagated to form fiber tears and fracture bridging areas. From the perspective of fracture mechanics, creating a continuous connection network facilitates stress transfer between individual fibrils. It prevents interfibrillar sliding. Therefore, the energy dissipation at the crack tip is not limited to the nearby crack tip but extends to the entire network. Likewise, nanofibrils extend their length through connections in a continuous network. Longer polymer fibers along the stress direction led to a higher toughness. The toughness of AGE-SC 36H hydrogel reaches 28.89 MJ·m⁻³ with a fracture energy of 411785.9 J m⁻².

Generally, soft materials exhibit a trade-off between stiffness and toughness (Γ~E^{-1/2}) according to the Lake-Thomas model, so there is a performance upper limit for state-of-the-art soft materials especially for hydrogel. The GE-SC 36H hydrogel demonstrates increased stiffness without becoming brittle, thanks to its hierarchical structure and multiscale synergistic enhancement. This unique combination of stiffness and toughness breaks the Lake-Thomas relationship, overcoming the traditional trade-off challenges faced by conventional hydrogels and even waterless elastomers (FIG. 4f). As a result, the GE-SC-36H hydrogel exhibits an extraordinary elastic modulus, along with exceptional toughness and strength, surpassing previously reported high-performance hydrogels and elastomers and successfully reconciling the mechanical property trade-off (FIG. 4g). Thus, the AGE-SC 36H hydrogel can incredibly high stability and adaptability in response to complex stress conditions, which makes it suitable for use in biomedical applications.

With a wide range of modulus and tissue-matched viscoelasticity, GE hydrogels effectively mimic the extracellular matrix (ECM), enabling stem cells to differentiate into various functional cells, from highly soft brain cells to extremely stiff chondrocytes. These hydrogels provide an ideal physiological environment and serve as a biocompatible framework for designing personalized medical solutions, establishing themselves as a promising material platform to enhance cell function and tissue regeneration.

The AGE-SC 36H hydrogel demonstrated a high degree of structural and mechanical similarity to tendon tissue, prompting further exploration of its potential as a tendon model for tissue regeneration. To evaluate the material's mechanical fit and predict vivo performance, its tensile properties were compared with those of healthy rat tendons. As shown in the FIG. 4h, the AGE-SC hydrogel exhibited significantly superior tensile properties compared to healthy tendons. Its maximum strain reached approximately 480%, far exceeding that of healthy tendons (~160%), and it maintained a stable response throughout the tensile process. This performance indicates that the AGE-SC hydrogel possesses excellent toughness and the ability to withstand large deformations, providing sufficient mechanical support to closely mimic the function of natural tendons.

### Example 7. Hydrogels Made of Other Polymers

Plant polysaccharides, including agar and carrageenan were selected for testing. The methods as disclosed in Examples 1-5 were used to produce the hierarchical hydrogels in this Example. Specifically, 15% Agar-1.5M Sodium Citrate 24H hydrogel and 15% Carrageenan-1.5M Sodium Citrate 24H hydrogel were evaluated, and both exhibited excellent mechanical properties (FIG. 5a). Notably, compared to the original 15% Agar and 15% Carrageenan hydrogels, the tensile strength of the 15% Agar-1.5M Sodium Citrate 24H and 15% Carrageenan-1.5M Sodium Citrate 24H hydrogels increased significantly to 7.8 MPa and 5.4 MPa, respectively, which are 180 times and 158 times higher than those of the original hydrogels (FIGS. 5(b) and (c)). Additionally, both the 15% Agar-1.5M Sodium Citrate 24H hydrogel and the 15% Carrageenan-1.5M Sodium Citrate 24H hydrogel exhibit ultra-high elastic moduli of 1.05 MPa and 1.558 MPa, respectively (FIGS. 5(d) and (e)). Overall, the programmed agar and carrageenan hydrogels display extraordinary elastic modulus, excellent toughness, and strength, outperforming most existing hydrogels and reconciling mechanical property trade-offs (FIG. 5f).

What is claimed is:

1. A cytocompatible hierarchical hydrogel, comprising: a cross-linked polymer, an added cation, and an added anion,
wherein the cation is selected from the group consisting of H⁺, NH₄⁺, Li⁺, Na⁺, K⁺, Rb⁺, Cs⁺, Mg²⁺, Ca²⁺, Zn²⁺, Cu²⁺, Fe²⁺, Sr²⁺, Ba²⁺, Mn²⁺, Ni²⁺, Fe³⁺, Al³⁺, Cr³⁺, Ti⁴⁺, and Zr⁴⁺;
wherein the anion is selected from the group consisting of OH⁻, CN⁻, SCN⁻, HCO₃⁻, HCOO⁻, CH₃COO⁻, F⁻, Cl⁻, Br⁻, I⁻, NO₃⁻, BF₄⁻, ClO₄⁻, SO₄²⁻, CO₃²⁻, SeO₄²⁻, WO₄²⁻, PO₄³⁻, SiO₄⁴⁻, Acetate (CH₃COO⁻), Propionate (C₂H₅COO⁻), Succinate (C₄H₆O₄²⁻), Fumarate (C₄H₄O₄²⁻), Oxalate (Oxalate, C₂O₄²⁻), Phosphate (H₂PO₄⁻ or HPO₄²⁻), Pyrophosphate (P₂O₇⁴⁻), Phosphonate (R—PO₃²⁻), p-Toluenesulfonic acid (TsO⁻), Methanesulfonic acid (CH₃SO₃⁻), Benzenesulfonic acid (BenzSO₃⁻), Citrate (C₆H₅O₇³⁻), Tartrate (C₄H₄O₆²⁻), Malate (C₄H₄O₅²⁻), Citrate (C₆H₅O₇³⁻), Isocitrate (C₆H₅O₇³⁻), Gluconate (C₆H₁₁O₇⁻), Oxalate (C₂O₄²⁻), Malonate (C₃H₄O₄²⁻), Succinate (C₄H₆O₄²⁻), Fumarate (C₄H₄O₄²⁻), Lactate (C₃H₅O₃⁻), Glycolate (C₂H₃O₃⁻), and Mandelate (C₆H₅CH(OH)COO⁻); and
wherein the hydrogel is characterized by at least one property presenting different values (anisotropic) in axis direction and transverse direction of the hydrogel, respectively.

2. The hydrogel of claim 1, wherein the cross-linked polymer is selected from gelatin, plant polysaccharides, animal polysaccharides, and a combination thereof.

3. The hydrogel of claim 2, wherein the anisotropic property of the hydrogel is tensile strength, Young's modules, fracture toughness, fracture energy, viscoelasticity, pore alignment, fibril orientation/polymer chain orientation, crystallinity, or tensile strain.

4. The hydrogel of claim 3, wherein the cation is Li⁺, Na⁺, Mg²⁺, K⁺, Ca²⁺, or a combination thereof, and wherein the anion is SO₄²⁻, CO₃²⁻, CH₃COO⁻, Cl⁻, NO₃⁻, C₆H₅O₇³⁻, or a combination thereof.

5. The hydrogel of claim 4, wherein the anisotropic property is tensile strength, which ranges from about 1 kPa (transverse direction) to about 100 MPa (axial direction).

6. The hydrogel of claim 5, wherein tensile strength of hydrogel ranges from 6±1 kPa (transverse direction) to 14.25±1 MPa (axial direction).

7. The hydrogel of claim 4, wherein the anisotropic property is Young's Modules, which ranges from about 1 kPa (transverse direction) to about 200 MPa (axial direction).

8. The hydrogel of claim 7, wherein Young's Modules of hydrogel ranges from 9.7±0.2 Kpa (transverse direction) to 3.595±0.120 MPa (axial direction).

9. The hydrogel of claim 4, wherein the anisotropic property is fracture toughness, which ranges from about 0.0005 MJ·m⁻³ (transverse direction) to about 200 MJ·m⁻³ (axial direction).

10. The hydrogel of claim 9, wherein fracture toughness of the hydrogel ranges from 0.00127±0.0005 (transverse direction) to 28.89±0.2 MJ·m⁻³ (axial direction).

11. The hydrogel of claim 4, wherein the anisotropic property is fracture energy which ranges from about 0.00001 MJ/m² (transverse direction) to about 20 MJ/m² (axial direction).

12. The hydrogel of claim 11, wherein fracture energy of the hydrogel ranges from about 65.8 J m⁻² (transverse direction) to about 413304.7 J m² (axial direction).

13. The hydrogel of claim 4, wherein the anisotropic property is viscoelasticity, which ranges from about 0.001 (transverse direction) to about 0.5 (axial direction).

14. The hydrogel of claim 13, wherein viscoelasticity of the hydrogel ranges from 0.0057±0.0012 (transverse direction) to 0.3867±0.016 (axial direction).

15. The hydrogel of claim 4, wherein the anisotropic property is crystallinity, which ranges from about 5% (transverse direction) to about 70% (axial direction).

16. The hydrogel of claim 15, wherein crystallinity of hydrogel ranges from about 9.32% (transverse direction) to about 30.33% (axial direction).

17. The hydrogel of claim 4, wherein the anisotropic property is fibril orientation/polymer chain orientation and wherein the fibril orientation/polymer chain orientation is characterized by inter-chain arrangement Herman orientation factor, which ranges from about 0.5 (transverse direction) to about 0.7 (axial direction).

18. The hydrogel of claim 4, further comprising pores, which has an average pore size ranging from 0.53±0.15 μm (transverse direction) to 10.91±1.96 μm (axial direction).

19. The hydrogel of claim 4, wherein the hydrogel is further characterized by porosity, which ranges from 10% (transverse direction) to 90% (axial direction) of the total volume of the hydrogel.

20. The hydrogel of claim 4, wherein the hydrogel is further characterized by tensile strain, which ranges from about 10% (transverse direction) to 500% (axial direction).

21. The hydrogel of claim 4, further comprising water, which ranges from about 70% to about 90% of hydrogel (weight/weight).

22. The hydrogel of claim 4, wherein the cation is sodium, and the anion is C₆H₅O₇³⁻ (citrate).
